SEQUENCE LISTING (59 sequences)

SEQ ID NO: 1 - DNA, 307 bp, Homo sapiens
ggcatgggaggctcagcaggtctgttcccttggcatgtgggacatcttgcagtggatgcagggagggaccatgcctctagcattctctgggaagaataaaggagaagtttatcttcccacctacctcccattgcctgtttttcattggtcaaaacttgtcccactaggaattaactccttcacatttccaagtttatatcatatgactgctccaactacatgggaccccaaaccccaaacctgatgtatgtatagtgttcctgtttcatccaagcctggaaatgaggggaagagccagaacctctag

SEQ ID NO: 2 - DNA, 1316 bp, Homo sapiens
gcttggtgctctggaaaaagaattagaattgcattcaaatagtcaggcttcaagttgcttctgcctctgtgtgaagcaaatagtaacaactcacttcgaaactcaggctcctggactgtaaatgaggataatgatactcaccctaccaacctcagggactgttctaagagcaaaatgaaatgagactgagctctgaaaatgcttaaatgcttttcaaagtagagtgatcgtgctttctgtagcatcaaaacagtcctctataaatgggcaaagtcaaggcatggactggatctgacaacagagaatggcttgagggctggaaaaagaaaaaaatgcaaatgccccaaatctctgcttcccttacagtgtggaaatcgccccataacacactgcaccaggtagggttaaattgatcccaaatgtttcaggaatttttaatgcccttcgaagagacatttccaattcttcatctgcccaaagtaatgctgctaacctactatctgtcagtttcctcagtgctttgaagatttttctaaagtcatctgacttggtttccctttgatttcaaattcctgcttattatatgtatgaccttgagcctttttaactcatctgagcctctctgtttcctcatctataaattgtgaataagaagacgttactgtaggattattagaaactttagaaataatacttttaataccagcatagtgcctgatccataggcatccaggacatgccagctaagatcattgattattgttgtcgtttttagtatcatacagtagaaagaacaacatctttgaaaccagacaatcccgcatttaaatcagctctgtcacttacgaaacgtgtaatcctgaataactgaagaaaactttttgaagttcaattttgagataatgatggatgatttcagactttgtgagaatttcagaggtatcggcagtaggttgaatggtgaaccctccaaaagatatatctatgttccaaactcagaatctgtgaatgtgacattatttgggaaaagggggttttgcagatataattaagttaagcatcctgagatgagatcatcctggattttctagatagggcctaaagccaataatgagtgtctaagacagaagaagacacaaagaagaagagacgggaatgtggtcacagaggcagagactggagcaatgcagccacaagccaaggaacacctggacccaccagaagctggaagaggaaagaagggatttctttttcttctagagccttgggagggagcatggccctgctgacaacttattttcagacttctagattct

SEQ ID NO: 3 - DNA, 937 bp, Homo sapiens
gtgagtactaggtggcttggcagtgtgtgtgtgtgtgtgtgtgtgtgtgtgtgtgtgtgtggttgaggggtggagagttaaggggacttctaaggtaagtccctgattagtcccagccagttgaactgtgttgggcaggtatggtggttgagtagttgaaggtcctagaagtggacagtggccacagtgtctaaggaggcacagaagatttgtgttgagataatgattcaagagaaaagaaggacccttcatacttcagtcagagaagaattcttttggtgggctatcatgctcttgggttaaattcttttctctgtaacattatttagaaaatcgcctccctctcaacaatgaagactgagtaccactggcttacagatatttctgtatattatctgagttagtagagtttgagtatcacatttctttccttccaattctatgatttgtcatcataaagttttggcatacgcagcccagtctttggaagaggggttgagaattgtgtaatatcaggtttagcattataaagtaacatctatcatctgaagatgagactataaacaccagtctggacataatagagcatgcttagcccacttgtttggaactacaaggaaaataaggtcatacaaaagagccctctgcccttaaaaaagaattctcaattgcaaatcacttctgagtaccagtggggaaatgcagtcatttattctcaagactgaaaagagaaaggagacctttttatcactttgtgtgtgtgtgtgtgtgtgtgtgtgtgtgtgtgtgtgagagagagagagagagagacagaaagaaagagcaggagagagagagagacagagagagagaatatgtgtgtgtgttacctacctaaagctaatggtagccaattttttcctttttttcttggaatttttttcttaatctaaaacag

SEQ ID NO: 4 - DNA, 302 bp, Homo sapiens
aacccccgcctagccccttcacagcaaatgcgctttttgccctgaagttgtgtgagaccgaccttaaactttctccgaccctatctcgggatgctacggctcaggtcgtggtggtttcgggaacctcaagacaacattccccagctgacctttgggaactttaactcgggtcgtcatgtacggaaaggtagtctatggggtacgcagaatggaaaagggcttccacatcagtcgtgtccgaggacgctctccaacttttgatgtccctggagagtttctgatatttccctggagagtttcag

SEQ ID NO: 5 - DNA, 1486 bp, Homo sapiens
aaaggatttcttccgacagacgtgaccccagaaggctgccgagggagactttaggacaagaggcgttgtttttgtgagaatgaatttttatactttaccccgaccccaccccctaccccccgcccaggtaaatccaagtaaatgattttctgaaggtcggtctcctagaaagcgtttaggctctggttttttgttttgtttctattttcgtgttttcccagttagggaaggaagaggagcctgtgcagagttgaactggggtggagaggtgccttgtaaagtatggtttccagagatggtgagacctcgtagctaagcgttgtttcctctgcacgtaactgtgagttgtgaacttgtggctcctgggggagcgggacagggcctggcagtgcgggggagggggcaaggaattcaggaagttggcgagaattcttcgtttttcattcttcgttgtaaattgcttctaaacacttctgaattttaaattgctaccgagaaggtccagaaaccgttctcatccaatttagtcacttttattacgttccactttgcctcccccaatcactgcggcggctcccagtttcagcgtggccgcatttggaaaaggctgaaggtagaaaactctggaatggagaggctggcgactggagcaggatccaggggctttaaattttaacctgaatgcgatcgaatgcgcgactcccagaagagatagccaggcgtccgcccggccgctcaacccccgccaggactcggaatgccttgcttttggttcttttcgtgagttccgaccaaatgtttaagtggactgggttttgcagaagctcatttgtaagcactgacggaaatgggttgtgggcatttgatttcctttgattccaacgttttgaaataatgtttctcggatttgctattcgaagcaattttctaaaaactcatatcttatttgctaaaaataaactctttggctcagtttgtgtccaggctgtgtacggcctccaaccctgagccccgaggagatcgagccctggcgcgagcctgatctctgtcctgtgtgtttctttgtatttgcaagtacaaggatgtctgcgtggatatccctggacactcgataccccccggggacctgcttttcccgcagctccgcagacgcggcgggagggcgcgctggccccggcgctgctgggacagaggtgagcagcggggtggacattgaccgattcgacaacgttcgggctctaagggctcgggaagggacccggcattttaccctccagattcccaatctatttttaaaaataatgttcaaaacatatgggaaaaataagtgattgctccttttcatggtcacagattgtatttattttttcagactggttttccaacctaacgagggaatgggtagtgaatgttgttgttgttgttgttgtttatgttggttaatttcgaaataaaactgtttaactcgtacagttgattcagacag

SEQ ID NO: 6 - DNA, 283 bp, Homo sapiens
gtaagtcacagataagtcactgcgaagagggggtgtcaccttccctggacccagcgctaaatgctgcttccccacccccacgttcatatacacaccccctcttcgctccctccaaacagttatcacaactctgtacaaactgatacggtccctagcgggcaaaagggactggaaatgaacgtcgcggggttatcagcgccgatctgtcagcggcaactcgcgcggccgcaggctcgtccgctgcaggagccgtgatatatgtctcgctccctttcgcttacag

SEQ ID NO: 7 - DNA, 339 bp, Homo sapiens
cattcttcgttgtaaattgcttctaaacacttctgaattttaaattgctaccgagaaggtccagaaaccgttctcatccaatttagtcacttttattacgttccactttgcctcccccaatcactgcggcggctcccagtttcagcgtggccgcatttggaaaaggctgaaggtagaaaactctggaatggagaggctggcgactggagcaggatccaggggctttaaattttaacctgaatgcgatcgaatgcgcgactcccagaagagatagccaggcgtccgcccggccgctcaacccccgccaggactcggaatgccttgcttttggttcttttc

SEQ ID NO: 8 - DNA, 330 bp, Homo sapiens
tacaaggatgtctgcgtggatatccctggacactcgataccccccggggacctgcttttcccgcagctccgcagacgcggcgggagggcgcgctggccccggcgctgctgggacagaggtgagcagcggggtggacattgaccgattcgacaacgttcgggctctaagggctcgggaagggacccggcattttaccctccagattcccaatctatttttaaaaataatgttcaaaacatatgggaaaaataagtgattgctccttttcatggtcacagattgtatttattttttcagactggttttccaacctaacgagggaatgggtag

SEQ ID NO: 9 - DNA, 295 bp, Homo sapiens
gtgagttccgaccaaatgtttaagtggactgggttttgcagaagctcatttgtaagcactgacggaaatgggttgtgggcatttgatttcctttgattccaacgttttgaaataatgtttctcggatttgctattcgaagcaattttctaaaaactcatatcttatttgctaaaaataaactctttggctcagtttgtgtccaggctgtgtacggcctccaaccctgagccccgaggagatcgagccctggcgcgagcctgatctctgtcctgtgtgtttctttgtatttgcaag

SEQ ID NO: 10 - DNA, 108 bp, Homo sapiens
gccgtgatatatgtctcgctccctttcgcttacagaaaggatttcttccgacagacgtgaccccagaaggctgccgagggagactttaggacaagaggcgttgttttt

SEQ ID NO: 11 - DNA, 119 bp, Homo sapiens
ttagggaaggaagaggagcctgtgcagagttgaactggggtggagaggtgccttgtaaagtatggtttccagagatggtgagacctcgtagctaagcgttgtttcctctgcacgtaact

SEQ ID NO: 12 - DNA, 200 bp, Homo sapiens
tttcagcgtggccgcatttggaaaaggctgaaggtagaaaactctggaatggagaggctggcgactggagcaggatccaggggctttaaattttaacctgaatgcgatcgaatgcgcgactcccagaagagatagccaggcgtccgcccggccgctcaacccccgccaggactcggaatgccttgcttttggttcttttc

SEQ ID NO: 13 - DNA, 54 bp, Homo sapiens
tacaaggatgtctgcgtggatatccctggacactcgataccccccggggacctg

SEQ ID NO: 14 - DNA, 148 bp, Homo sapiens
gtgagaatgaatttttatactttaccccgaccccaccccctaccccccgcccaggtaaatccaagtaaatgattttctgaaggtcggtctcctagaaagcgtttaggctctggttttttgttttgtttctattttcgtgttttcccag

SEQ ID NO: 15 - DNA, 240 bp, Homo sapiens
gtgagttgtgaacttgtggctcctgggggagcgggacagggcctggcagtgcgggggagggggcaaggaattcaggaagttggcgagaattcttcgtttttcattcttcgttgtaaattgcttctaaacacttctgaattttaaattgctaccgagaaggtccagaaaccgttctcatccaatttagtcacttttattacgttccactttgcctcccccaatcactgcggcggctcccag

SEQ ID NO: 16 - DNA, 295 bp, Homo sapiens; identical to SEQ ID NO: 9

SEQ ID NO: 17 - DNA, 60 bp, Homo sapiens
cgacagacgtgaccccagaaggctgccgagggagactttaggacaagaggcgttgttttt

SEQ ID NO: 18 - DNA, 134 bp, Homo sapiens
tttcagcgtggccgcatttggaaaaggctgaaggtagaaaactctggaatggagaggctggcgactggagcaggatccaggggctttaaattttaacctgaatgcgatcgaatgcgcgactcccagaagagata

SEQ ID NO: 19 - DNA, 507 bp, Homo sapiens
gtgagaatgaatttttatactttaccccgaccccaccccctaccccccgcccaggtaaatccaagtaaatgattttctgaaggtcggtctcctagaaagcgtttaggctctggttttttgttttgtttctattttcgtgttttcccagttagggaaggaagaggagcctgtgcagagttgaactggggtggagaggtgccttgtaaagtatggtttccagagatggtgagacctcgtagctaagcgttgtttcctctgcacgtaactgtgagttgtgaacttgtggctcctgggggagcgggacagggcctggcagtgcgggggagggggcaaggaattcaggaagttggcgagaattcttcgtttttcattcttcgttgtaaattgcttctaaacacttctgaattttaaattgctaccgagaaggtccagaaaccgttctcatccaatttagtcacttttattacgttccactttgcctcccccaatcactgcggcggctcccag

SEQ ID NO: 20 - DNA, 666 bp, Homo sapiens
tgatgtttgagccccagcatagatcagcatgccaggaagcagcccaccggacccacccctgatcatggcactggacattgttttccgtgcttttctcaggctggtttctgcctgtcccaaccctggccatggctgacaaggggcccagggacactggagcagagagacctgctggtccttggggtttcttgcaaggtaggactctacatcctcccttgggaactaattgatctgtcagtaaaatgtaaacttctgactccatgggtctgtgaatcagctgcctctaaagggaaatatcccaggagtgatgaaaacttgaagatcgaggattggatataagctcagaggtgaaggagggaaattcctcgggtcaactttagcggactgatccacggggtctgccaagagacaggagctcgagggttaaacccaagcactcgggagtcagatgtatgaatctgaatctcagcctctcggcttgctgtgtaatctcaggcaagtgacttttctgttctgtaaatcaggctgatagcgggggaacctctctcacagggctgctacaaagattgcatgagattttgctcaacaaatattagctgttggctccagagtttaatacctctgatcactacaagctttgaaggcaagataaaataacttcaacat

SEQ ID NO: 21 - DNA, 538 bp, Homo sapiens
agtgaatcatctgtgaacacttcctgtgaacgtgtgactatgattatggaacactctgttgcatcaagatggccagagattgtctcagagtccaggcagtgggaggaggggggatttgtttcctggctgggtggtcgtagggcatcaagggatgagcagctttggttgtctgggatttcagcagcttgaggggagctgtagggccccagagctgtcgctgtggctgtggtgtcagaaatgtaagcaaggtgcctccatggaggttagcgagggtgcccctctgaatgccgggtggaatgcaacctttcgcatggaggaggccctcagcagctgacctctgtcttccctgtgtagagggagggaggctgaggctggggctgagcgaggggtaaagtaagccacgtgtctcatcagctagcctgcagcaagcacagccctctgggagggctggttctatcctcctccttggtgtgaaatccatcaatctaatagggatccttattttcccagccaccaagtcacaatgagcaataatctc

SEQ ID NO: 22 - DNA, 13683 bp, Homo sapiens
gtgagtaccaggaggccttggtgaaaagcaagcctgccgggagataatgcctggctgggtcaggattcttggtgcttattgaccactgggcacttcagagcagaactcatcaaagcctctgtattgggacaagggctagggtggaggggaggattgggaagggagaggggagactgtccttctttctactaaaagttactggcaagagaatgggaattaaccaggcctggaaaagtaatttaggtattagaactttgaactctttttttaaaaacaaaagacaaaaacagctttattgggatataattgacaaataaaaattgtgtatatttaaggtgtacaacttgatgttttgatatatgtacacattatgacatgatcactaccatcaagctaattcacatatccatcacctcatacagtcactattttcttctttgtgtgtgtgagagatgagaacacttaagatctatcctcagccggatgtggtggctcacgtctataatcccagcactttgggaggctgaggcaggcgaatcacgaggtcaagagattgagaccatcctggccaacatggtgaaaccccatctctactaaaaatacaaaaattagcttggcgtggtggcgcgtgcctgtagtcctagctactcggaaggctgagccaggagaatcgcttgaaccggggaggcggaggttgcagtgagctgagatcgcaccactgcactccagcctgatgacagagtgagactccatctcaaaaaaaaaaactatcctcttagtaaatttccagtatataatacagaattgttaactatagtcccacgctatacattagatctccacaacttattcatattgcataactgaaactttgtaccctttgaccaacatgtccccattttcccgcccctgagctgctggcaacagcaattctactctctgcttctatgagttcaacttttttagatttcacatataagtgacattatacagtatttgtttttctgtatctggcttatttcacttaacataatgtcctccaggttcatccatgttgccgaaaatgtcgggattttttaaggctgaataatattgcattgtacgtatataccacatttttaaaatccatttatccattgaggtacatttgaatggtctaccctcctaaccaaggccaaccctttagcccaactaagctcttggaaaatgcccaaagttgctgcaaagcccctggctaaatccttctcatcctgagactccgcttcagcgttacctccccagggaggcctgggctgggtttgatccctaagtcaacccatacggcactttgcattgttaaaagaaatacttcagccaaattaaatttaaaggagtttaattgagcaatgaacagtcaggcagcccccaaatcacagcagattcagagatactccagcgcagccacatggtggaaggaaatttatggacagaaagaggaaagtgacgtacagaaaatagaacattttcatattgttcagtgagtgtcaggggccccaaagaatcatgattccacagcataatcacagctaacatgtatttggtgctctctatgtaccaggaatggcataagtgttgtctagtgaattcactcaacaactctgtgaaatagtttcccttctcacttggtttttagttaaaaaaaaaaaaatttgtagagatggggtctcactatgttgcccagcctggtctcaaattcctgggctcaagtgatcctcctgccttggactcccaaagtgttgggatgacagatgtgagccaccatgcccagaccccttcccacttttgcatctctaagctgccaccttgctgacccacacacagctgtgggtgaaatccaatgggctcagcagccatgagctaatagcactgctcctcctgcgtagagaaagatttattgtttcgaacctttgacttagcttagaaaacaaaatcagtaggccccactcttcaatgtttttaaggtatggtctcccaggtggattctgagacagctttcatctctagggttaattagggtctggccattgctcatgcgaagatagaagactaaaacacgcaaatctaatgccccatctttggcttccttgggaacagggggaggctggggtgggatggaatggaaaacacagagaagcaaatagcccacccaagaaagtgtgagccctcctgccttcactacctctggccaccaaaagagaccaaacaaaggggctaggggaaggaaagaattgggtgggggtaaaagtgggggtcttagtaaactctaaactcttagaaataaatgtagagttcacattccacctgggcttccaatgtccttctcagaggcagtcagggaccaccaaccagggagcgcgtatgagggctcagcttggttctagaacctttgtaaactcatgcttgctattttgatgatcacttacctgattttccagaggaaaactaacccagggaagcagggaggagaggcccatgagcctgtgttggaagaagaggaggagaggtcagcactccaccttcagccatgtcctagccagagaggggagtgacagctttctcaaagagaggaaactgggcagatccctgaggacttgatcaaggatgcaaggcaggggagcacaggggggctatgggctgagaggggcctggagggtggcagtggctgggcctagaggtgggctccgtaatgacacagggctggggcaatggaagcgcctagctcaggagcatggtggatgtgagtgtatatccgtccacccctagctagcaattcactagctgtgtgaccttcctctatctccactgattgtttccaagcctgagtttcctcacctgtgaaatggagataatccagcaattgtgacgagaaatgtgagaaaatgcatgtctgcagctattacagggtctgacctggagtaaaagctccatgaatgccggctgtcctgtggcctccccgacacgtcccccctgctggaaggctttcctcaccatctccctcataaacaaagccaaggtcaaggctgcagcccatggtgagagcagggagagcttgtgggaagttcactgccccgccatgcccctgaatggtgagcctctttctttgatgtgggctaacatccgtggactcagaggcccagaaagtgcgagacacgccaaggctcggcccctgggcgtctgtttcctgagcagcctgggacacaggttgccctggtgagatgacccacagtcagcaccctaggccttggcattatgcgggcagctggtatgagccctgcctcaccctgcacagaatttgccagcttcacagcattcctgccagcccagcctgctcccagacccagccaagtttcactcaggcagcctgcattcccattttgtgcgccttgtcctgcccagaggcaccggctgctgagggaagcatgaggtccttggaaaggagctgccccagcaggccggtgtgctgggagccctcagcctcttctacactcccgccacccccagccctgcatcctgccccaagctggctgcctgccagcttctcgggtctcatttacttactgctgtttgttttggaggcttctgggaagcaaggctccacctgaggaatctttggctgaggggacttgtgacacctgagactttgaactttcaagaaaacataaggctccgctggccacttccccgggtgtggggacaaatgtcactgcagtgccgtgggcttgcaccaggcccctcctcctctccaaagcagctaaggcagagctcatggacagggacctgcctgaagcagccgccatctgggggttgacggctgcctggtctcttgctctatctggaaaaagaaaccataagcatttcatatttatcttataattcatttggatttgttttcatataaaagtaatgtctaagggaggattagtcaagtaaattgtgcacatgttcttgtgatgacctattagcagatgccacagatttaccctagtttgagaaacaaaggtgagaagtatcttttgtagaccaggtgcggtggctcacacctgtaatcctagcactttggaaggccgaggtgggtggattacctgaggttgggagttcaagaccagcctggccaacatggtgaaactccgtctttactaaacaaaaaaaaaaaaaaaaaaattaactggacgtggaggcactcgcctgtagtcccagttactcgggaggctgaggcaggggaattgcttgaacccaggaggcagaggttacagtgagctgagattgtgccactgcactccagcgagattctgactcaaaaaaaaggagtatcttttggaaaacctctgttcaataattgacttttaaatgcacaaaaaattggaaatacaccaatttttacttttttttttttttttttttttttagagacaaggtcttgttctgttgcccaggctggagtgcagtgacaagatcatagttcactttggcctcaaacttctggcctcaagcaatctttccacctcagccttccaaacccctaagattacaggcatgagctacccagcccagacctcaatttttaaaaattgtaaaatgaacttttaaaatccctattaaggctgggcatggtggctgactcacgtctgtaatcccagcactttggaaggctgagacaggtagacaaggggtttgagactagcatgggcaacatggtaaaaccccatctctacaaaaaataaaaacaaaacaaaacaaaaacacaacaattagcccagcatagtggcatgtgcctgtagtcccagctacttgagaggctgaggtgggaggtttgattaaacctgggaggtcgaggctgcagtgagccaagatcatgccactgcactccagcctgggcaatgaggcaagaccctgtctcaaagaaaaaaaaatccctattaaatttggtagaattgtaatttaagcaggtttattatagtgagtttggaaagaataaagaaaaagaactaagttactccctcacacagagataacccttttggtgtacattttggtatatttaagatatcatacaatatatacttctttggtcatagttagcactcttatctaagtatttcttcccaatcaatcaattaatgttcagcaagattttttatttttatttttttgagatagagtatcgttctgtctcccattctggagtgcagtggcgcagtctcggctcactgcaacctcacctcctaggctgaagcgattctcatgcctcagcctactgagtagctgggactacaggcatgtgccaccatacccagctaatttttgtatttttagtagagatgggggtttcaccatgttgcccaggctgctctcaaactcctggcctcaaatgattcatctgcctcggcctcccaaagtgctggaattataggcatgagccaccgtgcccggctggtaagattattttttatttttagtggcagcatagtccactgtgtggacgaacaaaagttttgtttgttttttgagacagaatctcactctgtcacccaggctggtgtgcactggcatgatctcagctcatgcaacctctgtgattctcctgcctcagcctcctcagtagctgggattacaggtgcccaccaccatgcccagctaatttttgtattttcagtggagacggggtttcaccaggttggccaggctggtcttgaactcctgacctcaagcgatctgtccgcctcagcctcccaaagtgctgggatcacaggcgtgagccactgtgcacagcctaaaaattttttaaccgttcattttttgacatttaagttgttcctattttttgctgttataaaaaacattcaatagccacttttgcacaaacatctttgcacatttatctgagcacatacttaggataaacttcataaaattggaattgctgggtcaaaggataaggacattttaaggttgttggtacaccctgccttggctggtttcttcctttgcttctatgaaaaccattgccaccttgagatcagggacgtatttgcctaaattttcagtagtgctatgattttgtcattagatgtttagattataattcatctggactaatcataacactaataataatatgaggtaatacttattgggtggggactatgagttggacattgggctaagaaattcttatgcataatcttacttcacccctactattatgttcagggaactatttttatttcatcttacataggaagaaactaagattgtgagtttaacttgggatgaggcagggatctagctttatttttattttcctttctttctttcttccttcctttcttccttaccttctttccttcttttgtttccttctttctttctttctttctttcttcctttcttccttcctttctttttctttctttctttctttctctctctctgtttctttctttcctttcttttcttttttaaagacagggtctcactctgtcacctaggctggagtgcagtggcgagatctcagctcactgcagtcttgacctccctgggctcaagtgatcctcagcctcccaagtacctgcaaccacaggcacacgccccccatgcccggctagcttttcttgtgtgtgtgtatgtgggtttgtgtgtgtttgtgtgcgtgtattttgtagagatgggattttgccatgttgcccaggctagtagctttgtttttccatattattccaacatcatttattaaacaatgaatctttccatcactgatttgaatatcccattcagcatacactaccaatttctttttatacataggatctgttcctgggccttctcttcttttccactgagctgcctgtcctttttccatttcacacggttttttgttttttgtttaccactgaagctttaaaatgtattttactgtctgggaatgcagaaccaccctgttctttttctcccagaaattttatgattctattcatacataaatttatttccaggtgaattcattttgtcacattaaaagtaaatcttagatatttttattagaattgcataagacttatgcatttgggcaaatggcatttgacagtgttgagttctcggtgtggtgtgttttgccttttatttaatgtccctcagtgtcattcagcagattttttgttatgagttttgcacagttcctgtatacttcattccttggctttgctattgtaagtgggaactttttgcattatattttgtggccagttcaccctagtgtatagaaagctatgaaatgggtactagtgtttctcgtttacagatgaaggagtagaaattaagggcacagtgccaggtgagactctagttgaggacagggctattcagaggtgcggccgtgtctgaacctgcctcagtcataaccactctcttagaaggaagctgcccgtgctctccaaagacagtgtctggtcagacccagcttggtgtctgctgcctggcttggtgctcctggcttggtgtctgctgcctggacagttccagattcaagggcagccaaacatttctgggcagcaagctccctggcctgatcaagctctgggccatggctgtgccccgcaaaccccagaacccaataaaaggagtccccattaggtgggcatggctgcaggtgactgttgcacagtaggactttggaccacttatttgaaagctgctgttcttactgttcagcatcagagagaatttgatgaactctatggactctcagacaaatgccccaaatgctgttgacactttcagaggatgtatggacaaccccaaaacacacccaaggatcttgggttaaattaatattcctgtgttaaaacaatggcccagacagggattcctccaggtgggaggtgcccagggatggggtcacgttcagaggacagaagtccttgtgtctgtgtgactttggatgggtcacttccctctctgaatcgttattttctccatgagaacaatatctacccagtctagttatggtgttgtttgggaatcgtctggtatcctgtcttgtgacagtgctttgaaaactgcagttaagtaacacaaaggtaagaaatgactcatgccaggaaagggaatagcatgaggcccagctctgatatgcaaaggcctctcagattcttagagaatattattggctctcacaccaccagctgctcccagtgaacttggccttcgcccccaggaatggctgataaaggccttcactgctttccacttctgagctgatgggactgttttagctgacccagctaaaaccatgattaagtggctggatttggaaaagaagtgttggggctcatggttcacagatacgtttctgggttgttataaaaattaataattaggctgggtgcggtggctcactcctgtaatcccagcactttgggaggccgaagcaggaggaccacttgaggtcaggagctcaagactagcctggccaacatggcgaaaccctgcctctactaataatacaaaaattagccggatgtggtggctcatgcctgtaatctcagctactcaggaggctgagacaggagaatcgcttgaacctgggaggtggaggttgcagtgagctgagattgtgccactgcactccagtctgggtgacagagtgaaaatatgtctcaaaaaacaaaattaataattaatcctctcctgcatgctggaggggaactgccagcagcctccgagttatggagagcactcctggccaccaggctccccgtgagtcctggctccgtgtgagcccaggccagccctcctctctactgccctccactgggtccccatggtaaaataaggggctgcacttgatgaaagtgaatttgcaactgaaaaaatatgtaataataaagccagatcccttctaagagactttaaaggtcttccaacttgtcttcttcctgctcccagtagattatgtcccctgtcctcatgccccagcgtgaactttaggccctggacacatcagcttgcacacacatgtgtgtttgtgtgggggtgggtgggtgtgcatgtgtgtccttgggggaagctgggggaaaggggcaaaagagaaccctcactgggaacctttttctaaatacaaatggctgcccccttctccctgggaaatatctgaatggggtgagggatatactgctttggtcttccgaccatccagatccccatctccagactctccagccagagggcagggcagtagggggactggcaggactattttgaacgcctggttctcagaaccacctccctgcagtgctcctgttcagaggtcagaggtgtgcagcgtcctttcatcctctccttggagagcaagcctgatgaggcagggaatatctttctgactccccggttcctggctccatggcaggcatgcaacagaagctctcggacaaataaatgggaccttcccccatactttgcctaaaccttcttatgtcccttctctgctccctctttaccaatgtgggtatctcctctcctcccaacttctgctcaaattgtagcttctggggatgcctccagctgaacccccaggcctccccagatctgtggccccagccctgtgtccctgcatctttgctgatcccagccagaagctgccaggtatgcattttcatcaacagtagtgaggactatttattaatttatttatttttgagacagagtcttgctctgtcactcaggcttgagtgcaatggcctgaactcggctcactgcaacctctgcctcccaggctcaagagattcttgtgcctcagcctcccgagtagctgggattacaggcgtgtgccaccacgcccggctaatttttgtgtttttagtaaagacagggtttcaccatgttggccaggctggtcttgaattccagacctcaagtgattcatcctccttggcctcccaaagtgctgggattacaagtgtgagtcaccatgcccagccagtaaggactattcgttaagcagcattatagatcacctgtgtatgtctgctaaaaaaaaaaaaaaaaaaaaaagggcaccctctctacccagatgagcaaaggtattttttctgatacctgtaacaaatctaatattgccacaattgctacatgattagtgagagaagcatttgataggctaatcaattgattggtctggggtaaatattcagtgaatccaaagttcaactctaatgagccacttcacttctcctgggccctcttggtaatgtggtaccaggaactctggaagtcctcatggcattgctcaagggagtggtgggatgggtccccagctgctctttgcacagctctctgctctaggtgactgtctggtccatgagctgggctggcccagtctgtctctggcaatcctggggacatctgctgttgacatccttggtgagtgtaacctccagcttacacaacccattggacacctcctctgctctcaaaaactctgctgacccccaaagtatggttgtgactcccattaagcccaagacccacgtagaccctatgcccccagccagatcctcattggaaacccccatgggaacatacaagaaagtggatctcttccagttcctcccacattacgtggcatcaagggctttgggaatgtcaaaccaaatactcccctgtcctcagcagagcaccctcccctttcccaacgcccccccgccgccaccaccaggctaacgaagttctaaatgggggtcagggcacaaaatactctgtgtttgccttttctctcaatctattcaactgctcaccccacccaactcccaccaatccagtcctctaccctgggactttagcctgcagtaggaagatcagcccacgagtgtgaccacccaggtccccaacctcagcccatcttcttcccagtttcctgggcctggaggggagctctaatccctttcagttttagccttgatatccttaacatctttgccactagacagtgggccacggacgagctgcatcagtatccccagagcttattagaaatgtagactctcaagccccaccatggacttgcggaaccagggcttgtatgttaacaaggtgcccgggtgattcacatgcacattaaagtcagagaagcggtgctggcttgcagcacatcctggcagcgtgttctccttagcttatgtcagactcctggatctggtccgccaggcctggttcttaatatgttagtggaactcagcaaatttaggaaacccttctctctcttgtcaaagtttgacttctagatgctgcagactcaactatcttgcctccatgccttggatgtcactggctgcataaggactttgtctttctcttgacattcttgctgtaacaatcccagtcctcctcagtccattcttcttgaaaatggcactggggaaagaggcaaatatacagaatggagaaagagagaaacacatgaacatacttgaaaactactctcccagctacaatagcttttgctgacttgagcttcaagtgatgccagcaggctacatggagggtagaggtgagaaggggtggcacacacttcacagatgtcacagaggaggacgctccttctaccacactgtcccttgtctaaggacgcaaaggagaaaagccttgccttagcatccagagtggttcaatattacctgagcaagtgaactgcctttattttacaggcctctagggcataactatccatttgcccttttttgttttggccactaggaagcttagagacagatttgtggtccaacaacggtaagtgtatgatcatgtctaattctcatgcaggactgatccctggcttcaccctagcttgtcttcttcctccttgcttttaacatatgcattttattataaccatgtattcgtataagccactttaaatctctcctgaagccaagtgaaataactttgtaagtaggtactgttctaggtccttgcctatggcgtattccctcttgatctttgccattgttaacatccccattgcacagatgaggacactgaagcgcagagaggtgaaacaagcaagccaggctcagggcaacggtgagtgtccaagccagggtatctcagctctctgtgttcagagtttgggccccagaagggaccacctggatcctgtcttggcagcaggcaggccaacacggcgctgagagggagctgaggggaggtgtgggtcagatttctggcaagccctgaggctgtcagtgaaccgctgtggggtccccccagcatgtgctttggggccacaccctaatgggggtcttctttatgttatgaccactcctgcagttaatcaaccacaaggttgttttttgttgttgctgttttttctgtttgtttttttttttttttgagacagagtttcagtctgttggtcaagctggagtgcagtggcacaatcttggctcactgcaacctccatctcccaggttcaagtgactctcctgcctcagcctcccgagtagctgggattacaggcatgcaccaccacgcctggctaatttatgtatttttagtagacacggcatttcaccatgttggccaggctggtcttgaactcctggcctcaagtgatccacccgcctcggcctcccaaagtgctgggattacaggcatgagccaccgagcctggccttaagcacgagattcttaaggatagcactgggaccggtgattctgtgactggagctgattaactgctttgtgggaataagtaagggcttcaaagatgtgtgcccatctcggctgccaggccctgagttcttgaggtggaagctgactctccacctttctggctgatctaggcaggctagttccctgaagactcaaggtaagctgccttctagaatgaacttgggatgaaggtaaatgatgacacagggcagctgtctgaaaaagaaaggtttatttatctaaaccaacaagaaaggattctttttaattggaaaaacaattaaagaatagtgcatgtattcagatcctatttttcccagtgtgtttttgttgttgttgttgttgtttttgagatgaggtctcactctgtcgccaggctggaatgcagtggcgcaatcatagctcactgcaggctccacctcccaggctcaggcaatccttccacttcagcctcctgagtagctggcaccacaggcacacaccaccacgcccagctaattttttgcatttttgggtttcaccagttgcctaagctggtctcgaacttctgagctcaagtgatctgcccacctcagcctcccatagtgctgggattacaggtgtgagccattacacccagcctacgtgtgttttaaatacacatactcagatggatccactcagacacacacacagacccccatacacagagacactctatccccatcccgcaacacacacacacacacacacacacacacacacagagtctagaaaggaccaccaaacaccaaagtgttaatagctacttccgaaagcagagatagaaactaggctaatgggtgaggaatgtttaccttttaattttcactatatatactttccatattgttagaactttttacaacgatgttgtcacttcatgatttaactgttttgttttgttttgttttttaatag

SEQ ID NO: 23 - DNA, 82 bp, Homo sapiens
gtcccaaccctggccatggctgacaaggggcccagggacactggagcagagagacctgctggtccttggggtttcttgcaag

SEQ ID NO: 24 - DNA, 142 bp, Homo sapiens
gctgatagcgggggaacctctctcacagggctgctacaaagattgcatgagattttgctcaacaaatattagctgttggctccagagtttaatacctctgatcactacaagctttgaaggcaagataaaataacttcaacat

SEQ ID NO: 25 - DNA, 133 bp, Homo sapiens
agtgaatcatctgtgaacacttcctgtgaacgtgtgactatgattatggaacactctgttgcatcaagatggccagagattgtctcagagtccaggcagtgggaggaggggggatttgtttcctggctgggtg

SEQ ID NO: 26 - DNA, 329 bp, Homo sapiens
gtaggactctacatcctcccttgggaactaattgatctgtcagtaaaatgtaaacttctgactccatgggtctgtgaatcagctgcctctaaagggaaatatcccaggagtgatgaaaacttgaagatcgaggattggatataagctcagaggtgaaggagggaaattcctcgggtcaactttagcggactgatccacggggtctgccaagagacaggagctcgagggttaaacccaagcactcgggagtcagatgtatgaatctgaatctcagcctctcggcttgctgtgtaatctcaggcaagtgacttttctgttctgtaaatcag

SEQ ID NO: 27 - DNA, 13683 bp, Homo sapiens; identical to SEQ ID NO: 22

SEQ ID NO: 28 - DNA, 470 bp, Homo sapiens
gcgagccacgggcctcgctgcgggttcaagtgcgtccgggtttggacgtcgcggctccaggagtgtgctctctctcctgcccatcccctccccggagttcagcgcatccggggccccggggacccctcttcccggccctcctggccgcgctggcccgcccgcatggggccgaggttcctggccgacgcgcgagggcgcgggcgggtaccgggttcccgtttttcccaggccccgatccccgcccacgcacgaggcccccggcccacgcacgaggcgcccacgcccatcgtggaggctccgcctggaaaagaagtccgacttcctctccaagctgctccgaggggaatggggaatcggcaggaaatgacccgaactgccagcctgcgcctttgcagccggccctcgctttgctgaagacgagcagctcccaccaaagtcttgcctccccttaccccgaaagccccgctcag

SEQ ID NO: 29 - DNA, 93 bp, Homo sapiens
ttcaatatttcaagtgaaaatctgcctgttcgaaaagaaaatattcacaaatgagcagtgtattagaagacaactgaattgcttctaagaatg

SEQ ID NO: 30 - DNA, 844 bp, Homo sapiens
ggtctcactctgccatcacccaggctggagtgtggtggcaccatcatggctcactgcagccttgaactcctgagctcaagtcatcctcccacctcagccttccgagtagctggaactgcaggtgtgagccaccatgcctggctgatttgtctttttaaattttttatagagaccgagtctcaactatattgtccaggctggaaaagaacttctttccctccaagtgataaccttttccagacaagtcagcttaaaaactgacccaaaggccctgaagtacacagttttcttggagaaagaaaaaaataagtgaaaaggacttgctgacttgctaattcattttggggcctgtgggtaacatcagaatggcaaagcacttggggcttttttctaatttgtatttttttaatcttctgagaaatgacttttatatgccaagattcattccatgattagtttttatgaccaagttatgaacgcttttgaatatggggacctagaatagaaatgctaacctacctgcaaggagagctctgtgattggcatccccatcatgctgtaactgacacaaaataaacttagggtaattgccgcatagtgtaactttaatgtgtgagcatactgtaacatctgacaagcttcttgatctagatcagcaatgtgtaacccattcattgttgtcactctcaacaaggccttatggtcaaacatcatcactctcactaaagagatagtttactgaaatatactgagattttgaaactggagtcaagaggtagcacactgttttatgttgatccatttaatggtccttgacagccaataaaaagacactggaagaata

SEQ ID NO: 31 - DNA, 4336 bp, Homo sapiens
gtttgtaaagaggggtgcgagatctgaaaagtatttgtcttctagggagagagatgttggtaaggggcccagagaaagacgggtcctttttagctcatcaacgcgccacgtcttgctttgcagctccaaaatgatgccagcgctgctgtccgaagggtcaatattaatgcgttaagtgaaatgggaaataaatggacagtggcacatttcgtggcatcatttttacaaggggatgtcgaaagttttaagctgcatttaagaattagccacatcttgcctgaaagttaatattttttgataaactcgttaataaagcctagaagaggtaagttaaagttagttaccacgtagatcctccagtctcttgtagagacttgcgcaggttactaggatgaggacgagttgtatttttaattttctagactaagtgcaaaggcattgagttttgaggtggcagggttaatgttcaagggcaaatggaaccgccaaggatctcttgaaaagaattcacttactattaatacttaagcctgaaataattataattaatttacagtgggaattatatgcccatgttttagacttttttgttatattagctatatgaaaagggatgttctttagtgattaaaagaaaaaaccacctgtttattgtcatttgcaatttgaaatagccgacagcagtaaggctagctacaaaagctaacaagtcaacttcagtgtggtttgacatttactgtcaaagctttgagctctaagaaaaacagaatttaaaataatgtatataaataataaaatttcattgtgactcaatgtccctgcccctggagaagggacagatgacactcagtgactggcttggaaccattccccctgcagatgcatttgcggatacagtgggccagggctgcctctgagtatgtacagcaagtgcttatggacttacagaaacaaatgttttactatttaggattttctcaatagtttacactaaaagttttgcatgtagaaatgtgggataaatatttctttgaaaggaaaagtttcgaaaggaaactttatacttgtatttttttgtgtgttcacatttctaagttcatctgtctttcaaaaaacagattttcataaatattaaaatattgcaagtattatttttaattcttttgagtgccatgtttgctgtaagtagttttttcttttaagtaagattaatataatcctgtctaaggagtttaatagagagaacaggatttccaagcctataaatgcagtctatataaaatagtgggatcaaagttttttgttaagaaacttgagaatttatacattttaaatgaagatagaagcacgttttaaaagtagtttttccacaccatttccctccccccaaaagaattcagaaactatattccatagtctaaaaatggcataatcagatgtaaaaattattaccataatatatagttttctcaagatggtggatactttttaatatattatgaagagttatattctcagcccactgtgtgtttgagggaatggaaaagacaagatgtttctaaacatattttactactgaaatccataagactattaagctgtgcaggtaattgtgttctaagtggtagaagtatttttccaactgctttttaaactttgaaaaatttatatgtattttaattagcacttttcttttaaaggttccaaatatttagctatatcagctatgtgataaaaattattggaatttgttccctactactttctgcataaacaattgagaaatattttatactaatctttttatatacaggggataaatttgaacatgtgtcttgttggtgtaataaacaatccaaattcatttctttcttgaaaacttggcatcgatcttcatcttattttttatgcacacaatttggacatgccagtttgtaggcaagttttaagacagtgcaagttttatgtggaagaatgtatattaaggaatgaatagctaatagccaaaattaaatcaggaggtgtggcattcactcctttggatgattattttaaagatagttttgcactcaggaaggattttcttatgctctgtatttatgaatatctaaattatcctgccttgcaacttcaaaactcaataaccaaataccaaatttatgtattttcagtgtgactaaggctaggaatatgagaagcagattggttttataaaagaaagcaagcatttacaattgtattttaaaatataatcctaatttcagtttctgacagaaatcttgtggtcagctcatcagtttttcagatcagtttctaaatagcatataaaaactaccttttccacatcagaagttcatatttaggtttccttaactgcaaaatcactttaaaagagagtgttttatgtgaagggccatttcatgtgttggttccatcgattacatatgaatgtataaaaatcttgtttaaagaccagtaaaagtgaggaaattccaagtcaggctggctaattccattcataattcatcccgagtacctgactgaggaaacttgcaaacagtttatagctatacatacaaaataagttgacgtgctttgtaagctggaagcagagttaagtttgacctccctgcttggaaggtcttaaattaataaaattatggaatatggttaagcttttcagtttcccaagaattttccaatatcgatgctttttggatttttgcattttgaaaatgtaagcatggaaggacaacttcttttgtgctgagatgaagttagcttttacccatccatttccttcccacttaacacatttgttcagtaattcttcgagtgctgggggacagaggccctgccttgagaaaagcatatgccatttctctgcccccttcaaccatccccacccccagccctccctttatttcaagccagttggacaagtatttagcattgacacatgtttcaaactgctgcatccacagccctaggtagttttggggattggcaggaaatccatctagactttgccttctgagctgggtaaaactactaggaagtcctatgccttgatattgtggcacttctaggctgtctttccaattttaagcaatccatcagttgttttctaagactgcactgtagactgatgccttctcttagttttgatgttcagaagattttttaaacatgttcatagatttcttaaacattctgagtaattgtaattttaaactagactaattttagggttgaaaactgtcccagagagagtccttacataataacttcaatctgatgtgaactcgaatgactttttaaaaataagatgttgaaaatgtgggtttctggtggaaaaggctgccattcacttagagtgggatccttaggaccagttaatatccaccagacatgcttcaataaactggaacaagcctgatgtggaattactgactccccattttcctgaatgtaggtcattttgtgtccttacaatttaacgtttgaagggatttcctgtctttgaagtgcaacagtttgtcacctgcttggctagctgccttaacagttggggctcctttcctacattgaaagacttagtgactccatgttcccgggctagagtttcccagctcactgaatgcatgatacctgcacctctgggactctcctctacaagttagtgcctgcagatcccctgttccaagcttcatttctctaggtcaaagagcctaaaagaaaagcccgggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtagtggcgggcgcctgtagtcccagctccttgggaggctgaggcaggagaatggcgtgaacccgggaggcggagcttgcagtgagccgagatcccgccactgcactccagcctgggcgacagagcgagactccgtctcaaaaaaaaaaaaaaaaaaagaaaagaaaagcccggagccatacactcacctggttgaagccataaagaggtaagctttcataacgtaggctatatatcaaaattcatagctttttagaaccggagtaactagtgctcaggcattagagtctcaggctagagcaaaacacagcaactggtgccaaatgtacgctagtggctgaaacccatttaagtaataaacaaacaaatgtccttttaagaatgtgctattatttttatttaagaatgcatatgactttatcgccccttatgatgtgaatcttacag

SEQ ID NO: 32 - DNA, 2600 bp, Homo sapiens
gtgggtatgctaatttttaccaaatgatacaacatgtgcatgccaaagctctttgtaaactataacacacctccaaatgaaagcaatttgcatatattttaataaacttgtctgcctttaccacatccaccaactaatccttatgctcccactgcaaagactctcctctttttggagaggaacaccaattgtttgaaagcctggactttcaaacaatcttaaggcatcttcatagtatccgtccttaagagtgctttcatcttaatattaggtaaacatttagaagattatgaactttggggactttcttttcagagaactgaacacgtgtatataaaattacaatatcctgataagtgaggggaagaaaataaaaataggtgtcaatacagttatggcttaagatcttgatgaaattgagaagaaagtagaaatcaaatgcatcaaaggaagaacggtcagactgaattctgtttgtaattttttcaaaatgaactgaaatgcagtttttctgactatatgttttcttttatggacattaatcactttgattcatttaatttctctggaagaaactgtagcttttgtaacagaatctgctctattgggttctgtatgggcacctaccggtttgctttctagccagtatcatgtgaattgtctagagaaaatgcctttagcaaacaccttcaaaaagcatccattccaagggcacacctcttgtgtctctcacaaagcaagtggtactatgtctagccaaggaggcagactcagattcatgcatttatctcaggttttttggtgaccttcttcctagatcctgctcttggcaactgtaagccaagtgtacataagtattagaatggattctctaggtaccaagtgaaaattctatctctagtcacatgctcctgcacctttcatgtgggaaaaatgttccagtctttccttgattacatcacagatgatacgagaacaggacatgggacagggaactgctaaacagctggatatctctggaaactcagagcacttaaaaacactttgaagccatgcagctgctctttttttttttttaaattgtcttcagtgaaactccaaaaccccaataatatctaccttaaaagcatcaggagtattttaattgggcaataaccatccatattttccttctgataaattaaaatgaattaagaatgaaattttatgatgattataaagtatattactgacccaagggacaagaacaattatgaaaaatatgtcctacatatttggcatgaatatgaatttagactttaaaccattcatcttaaatgctcaagcatcttattaaagcttataaaataatattcacataataacagagtaacttataatcatgattgatattatatcatcaatattgcagcaggctttgtgccaaatactgaaattgacttggtctttgcctgttggacttagaataaaaataaatacattattaaaaagatgaaacatggtaaaagattttgtgcatttgagatttttttttctttctccaattaaaattattcaccatgagaacgaaaggtgatttttgaaaaacctggaaaacctagtgagttggaattgtactactaaatgttggaagatctgatcaaaatttaactttaatagccagcagctgtttgaggacaagttcaccttacatgaatgtgggtggtcatttgagggtaacccaccaagcttagcctagcggtggccaggccctaaaataggagtcatcttggagagcaagagcatttctgtatttgagttaatgctacccaccagcaatcaagatcaattaactcttgtagtaaactttttaaatgaattggagatgactacagaaattgccaaagacaatgtgggcaaaaatggatgggctaagtgaaggggctgcctgtatcagcatttacttaggcccagccccacaaagccctagcagttgccattcagacacctgtatgttaacattaagacctttagagcatatgtttaactgtgagtattttcttccacataggtattggcactttttagcatgacctacctttgtgtgtcatgctaaaaagcatatatgactaatagaacacacacatttaacttattatgggcctgggtttcccgggtttcagcttcagtgtatgtcccctggcaggtttcttaactggtctgagcctaagtttcctcatttataaaacaaaggcagtaagtggtgcctttttgcaatattaaaattagtattaaatggcataacgttcacaaaatatctggaacagtttgccatcagtaaatatctgctactgcagttgcaagggaaaaacaagattgaatctttgttctcagtttctcattatataaaattttagagattatttaaagaaaagagagagagcatctaaatgaacagtttcaatagcatcttgtttatattcagaaatacagccctgaactcaaacttgaaacttcagtgttagccaaccttccctcctcccatcaccacctgaaggaaaatatctttgtgtagcacaaaagaacttaattctaattctttttttttccttttaaaacag

SEQ ID NO: 33 - DNA, 101 bp, Homo sapiens
cgaactgccagcctgcgcctttgcagccggccctcgctttgctgaagacgagcagctcccaccaaagtcttgcctccccttaccccgaaagccccgctcag

SEQ ID NO: 34 - DNA, 376 bp, Homo sapiens
gtcaaagagcctaaaagaaaagcccgggccgggcgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcgtagtggcgggcgcctgtagtcccagctccttgggaggctgaggcaggagaatggcgtgaacccgggaggcggagcttgcagtgagccgagatcccgccactgcactccagcctgggcgacagagcgagactccgtctcaaaaaaaaaaaaaaaaaaagaaaagaaaagcccggagccatacactcacctggttgaagccataaagag

SEQ ID NO: 35 - DNA, 93 bp, Homo sapiens; identical to SEQ ID NO: 29

SEQ ID NO: 36 - DNA, 268 bp, Homo sapiens
ggtctcactctgccatcacccaggctggagtgtggtggcaccatcatggctcactgcagccttgaactcctgagctcaagtcatcctcccacctcagccttccgagtagctggaactgcaggtgtgagccaccatgcctggctgatttgtctttttaaattttttatagagaccgagtctcaactatattgtccaggctggaaaagaacttctttccctccaagtgataaccttttccagacaagtcagcttaaaaactgacccaaag

SEQ ID NO: 37 - DNA, 3721 bp, Homo sapiens
gtttgtaaagaggggtgcgagatctgaaaagtatttgtcttctagggagagagatgttggtaaggggcccagagaaagacgggtcctttttagctcatcaacgcgccacgtcttgctttgcagctccaaaatgatgccagcgctgctgtccgaagggtcaatattaatgcgttaagtgaaatgggaaataaatggacagtggcacatttcgtggcatcatttttacaaggggatgtcgaaagttttaagctgcatttaagaattagccacatcttgcctgaaagttaatattttttgataaactcgttaataaagcctagaagaggtaagttaaagttagttaccacgtagatcctccagtctcttgtagagacttgcgcaggttactaggatgaggacgagttgtatttttaattttctagactaagtgcaaaggcattgagttttgaggtggcagggttaatgttcaagggcaaatggaaccgccaaggatctcttgaaaagaattcacttactattaatacttaagcctgaaataattataattaatttacagtgggaattatatgcccatgttttagacttttttgttatattagctatatgaaaagggatgttctttagtgattaaaagaaaaaaccacctgtttattgtcatttgcaatttgaaatagccgacagcagtaaggctagctacaaaagctaacaagtcaacttcagtgtggtttgacatttactgtcaaagctttgagctctaagaaaaacagaatttaaaataatgtatataaataataaaatttcattgtgactcaatgtccctgcccctggagaagggacagatgacactcagtgactggcttggaaccattccccctgcagatgcatttgcggatacagtgggccagggctgcctctgagtatgtacagcaagtgcttatggacttacagaaacaaatgttttactatttaggattttctcaatagtttacactaaaagttttgcatgtagaaatgtgggataaatatttctttgaaaggaaaagtttcgaaaggaaactttatacttgtatttttttgtgtgttcacatttctaagttcatctgtctttcaaaaaacagattttcataaatattaaaatattgcaagtattatttttaattcttttgagtgccatgtttgctgtaagtagttttttcttttaagtaagattaatataatcctgtctaaggagtttaatagagagaacaggatttccaagcctataaatgcagtctatataaaatagtgggatcaaagttttttgttaagaaacttgagaatttatacattttaaatgaagatagaagcacgttttaaaagtagtttttccacaccatttccctccccccaaaagaattcagaaactatattccatagtctaaaaatggcataatcagatgtaaaaattattaccataatatatagttttctcaagatggtggatactttttaatatattatgaagagttatattctcagcccactgtgtgtttgagggaatggaaaagacaagatgtttctaaacatattttactactgaaatccataagactattaagctgtgcaggtaattgtgttctaagtggtagaagtatttttccaactgctttttaaactttgaaaaatttatatgtattttaattagcacttttcttttaaaggttccaaatatttagctatatcagctatgtgataaaaattattggaatttgttccctactactttctgcataaacaattgagaaatattttatactaatctttttatatacaggggataaatttgaacatgtgtcttgttggtgtaataaacaatccaaattcatttctttcttgaaaacttggcatcgatcttcatcttattttttatgcacacaatttggacatgccagtttgtaggcaagttttaagacagtgcaagttttatgtggaagaatgtatattaaggaatgaatagctaatagccaaaattaaatcaggaggtgtggcattcactcctttggatgattattttaaagatagttttgcactcaggaaggattttcttatgctctgtatttatgaatatctaaattatcctgccttgcaacttcaaaactcaataaccaaataccaaatttatgtattttcagtgtgactaaggctaggaatatgagaagcagattggttttataaaagaaagcaagcatttacaattgtattttaaaatataatcctaatttcagtttctgacagaaatcttgtggtcagctcatcagtttttcagatcagtttctaaatagcatataaaaactaccttttccacatcagaagttcatatttaggtttccttaactgcaaaatcactttaaaagagagtgttttatgtgaagggccatttcatgtgttggttccatcgattacatatgaatgtataaaaatcttgtttaaagaccagtaaaagtgaggaaattccaagtcaggctggctaattccattcataattcatcccgagtacctgactgaggaaacttgcaaacagtttatagctatacatacaaaataagttgacgtgctttgtaagctggaagcagagttaagtttgacctccctgcttggaaggtcttaaattaataaaattatggaatatggttaagcttttcagtttcccaagaattttccaatatcgatgctttttggatttttgcattttgaaaatgtaagcatggaaggacaacttcttttgtgctgagatgaagttagcttttacccatccatttccttcccacttaacacatttgttcagtaattcttcgagtgctgggggacagaggccctgccttgagaaaagcatatgccatttctctgcccccttcaaccatccccacccccagccctccctttatttcaagccagttggacaagtatttagcattgacacatgtttcaaactgctgcatccacagccctaggtagttttggggattggcaggaaatccatctagactttgccttctgagctgggtaaaactactaggaagtcctatgccttgatattgtggcacttctaggctgtctttccaattttaagcaatccatcagttgttttctaagactgcactgtagactgatgccttctcttagttttgatgttcagaagattttttaaacatgttcatagatttcttaaacattctgagtaattgtaattttaaactagactaattttagggttgaaaactgtcccagagagagtccttacataataacttcaatctgatgtgaactcgaatgactttttaaaaataagatgttgaaaatgtgggtttctggtggaaaaggctgccattcacttagagtgggatccttaggaccagttaatatccaccagacatgcttcaataaactggaacaagcctgatgtggaattactgactccccattttcctgaatgtaggtcattttgtgtccttacaatttaacgtttgaagggatttcctgtctttgaagtgcaacagtttgtcacctgcttggctagctgccttaacagttggggctcctttcctacattgaaagacttagtgactccatgttcccgggctagagtttcccagctcactgaatgcatgatacctgcacctctgggactctcctctacaagttagtgcctgcagatcccctgttccaagcttcatttctctag

SEQ ID NO: 38 - DNA, 239 bp, Homo sapiens
gtaagctttcataacgtaggctatatatcaaaattcatagctttttagaaccggagtaactagtgctcaggcattagagtctcaggctagagcaaaacacagcaactggtgccaaatgtacgctagtggctgaaacccatttaagtaataaacaaacaaatgtccttttaagaatgtgctattatttttatttaagaatgcatatgactttatcgccccttatgatgtgaatcttacag

SEQ ID NO: 39 - DNA, 2600 bp, Homo sapiens; identical to SEQ ID NO: 32

SEQ ID NO: 40 - DNA, 1907 bp, Homo sapiens
ttcccgcactcaggcgccggttgaggctgcgctcctggcgaaggacaggtgctcctttcagccggttaacgttcagctcaagaccggattaagtctcttgctctccaaataaaattgtaaaggtttggaaatatagaaccacatttttaccaaaaacctactagagagcattcgtcagcttgcccttttcgccccgaaaaacgactttttcattcacagcctgtctcttcctctttttaaatttatgccgacagaggaagaagctgatatttaacaaataacaagaggttgcgataaatagaatttaaggtgggaaagcagccgccaaactgacaaatcgtcttcgagaccgacaagaaaatagtagtccggaatttggctaatggatggagaaggtggtcgtttttcatagacccttacggagaatatctagtgaagctaaagagatcaccataaacttatgcattcaaacgcatttctggaacagaggtcgtaaccgattttgtttccatcatcagtcctgaacagttgattatttatataatcaactgttatttatataaaggtaataaaaatattacctttcctcctcctttacaatgaatatctgttagaacacctgccagtgtaacaatcagactacaaggctacaggattatgattactgtctcggatgatgagcccggggtcctacaaatcatgacccccgccggcccctcattctgcccatatttgccaaacgtctgcatcagtaatttgaataatttaaaatagttccaaaacagcaaagctcagcagttacgcaaagaaacaagaatggaaaacatcccctcaggcacagagaagtgtccacccctttgggtggaggaattatacaaatcaggagaaagataaggacaatagcagttcatcacgatttatcccgcctctgcaatgttctagacaaattaactcggagcgctccaatggacccgcatttgtctctttactattcaatcgctgtaagatatacggagactgcatcactttaaatgggagtatcaattgctttgtggttatgctggattttgtgttattgctcagtcactaaagtaagaaaaatctatatggcttgtcaaatttatgcctattgtatttcgccccctgctggtttagctcgtgtgcaagcttgaatttgtgtgtgtgtagaaactagaaattgattttaaacgaacacatgcttgttttcattttacagctctactaaacagaaatatcaaaataagaacatatgactagaccttaaagcagaccctacactaagaacatgtttaaactaaatgaatttaaactgcactccagcctgcgcgacagagtgaaaccctgtctaaaaaaaaattaaattaaattgcacaccccccatcagcttacaattttgtaatagggaaattgagtacctatttggccaactaacttgtaattggtaatcttacatgtaattccgaagacagtgccctaatgctcatgacattttattacaaagggaaagtatttgagtgtttttaaggtgataaatctgtgggaaatataaagtccatttcatttggagtcaatgatagtattgtttttatttaccttttaaaaattatggccataactgcattacctaagatttggcatcaagaaaagaagaagaaaaaaattaccagttaacatttgttgtatttacttctagtttttttttcctgtgcgtactttttacaaacttgtaatcataatttaagtagattttgtgtcccttttgtgttgtaagcatttccacccatcgtttaatgtaaaaccttaattttaattggctgcttagtagtcaatggaatgaccactatatggatagttttcagtattataaaataatacttagatgaaca

SEQ ID NO: 41 - DNA, 2037 bp, Homo sapiens
atagaagctgtattacagaaacttttaatctcttttctatgtcagtaaggggatgctgaaataaactgtctagaaaaacaagaagcttctgtcacacccaggccactttcggcagagtggtccagagcggccagggcacggccagctgctttgcatctgcattcccgtttcattaccttttgtgttgtgggccaacaggagtgctacgtagtgggggctggagatttgtccaggcaggaggtgggcaagtggctgtcttctctgcagaatacctgcactgctccatctgaccagcagtggcaacagccacaaccagttctgcatctagctcctgcccagacctttggctgccagaatctagaaccatggctgaccaaagcgagacttccgagtaaggttcgttaacaggcatcctgctgaattagaaggaaacaggaacgaatccaaagcaaacggggctaaacttccctcgaggacatttcctgagctgcaacatgggaagcgacagcaccgaagctggtgagctggtgcaactgcacacaccttttctctggtttttagtactcacaggtcatcctggggagaccgctcatgggctatgggcagcccatgcctggattttttggtgaatgagtctgaaaagcagtgaccaggtgtgttgagggcctccccttgcctgcacctttttatctctgagtgactgagggctgccaaggccagacaacgcacatgtgtcctggatcctcccctggcctggggcagcagcagcagcagcagctgggcttggggtgagtaggctggcttgaggaaggggctgcccacaagcagggctcatttcctgggtcagggcctgtcagactcaaagtcagctcttcagcattctgtttagaaaatgatgatgcagttcagtgagaaagaaagctcaggataaaatatgttttctggaggggaaatactcaagtgttctaagccaggaacagagactctcaagagaggaattctctggcattctctgccttggataaatcatggattaaacatgagcatccttagaataattttatttttgtatttcactttacactgtaatgcaaacagcttttatgttttctttgacgaagaatcataattgagtcactttaggtcttttagctggaagcatttcaccctaaatgaaaagttaatgcattttttacaaggattaacatgctaactacaacttgccctttcagcagatgacaataagggattctgaaaagtttggactgtctttttctttttgtatacaaataaagcagcagatcctttctctgtgaaggttgttgcagagctcccgtaccagcttataaaatgcggctgtttaagcaggacttgggtgggcagcagaattctgcacatgacgagtggctgcagcttagaaagcaaatttcatctgattccagtactgtgattttaaggaaacggtaaaactcaaagtgccagtaatgccatcaaggctcagtaacatgccatttcctcagcagctaaagacaaaaagaattattttggtgaggggatagaaatcctttcgctttgcagtgacacccactttttcttagcttggcttgggagtaaggagattggagtgtggggggacattagaaactcatttaaggcaaactgaaatctccttcatcagcagtcttcctgctggtgtagccacagcagaactccgaggcttgcaggctgggctgtctccttgctgcctggcttgggtgtttatttgggcaatgcaagctgacattgtgaggtgttgccgatgcccaggccagctagcagggagccctcccagggctggtagaaacctatgtgtatatatattctcaaatttaaatagattgtacatagtgaaatgtaaatgacatgtaaaacagcaatctctatttttcttaactattatatagcagtatatatttgttgaacatgcttgtatactcttcattaaccattttaatattttgtacagataagttgattaaatattttattcataaaacc

SEQ ID NO: 42 - DNA, 11210 bp, Homo sapiens
ctcgcccccccggagccgacaaatggccgcggcggcctcaggtccggcgtcactcagacaagggggcgggtggaggaggaagcggccaagtccacagtcctgctcccgtgccgccgagcaccgcccacctcagctgaccagccctgtccgctactaggcctcgcctagaggagggaaaggccgtggcgtccgctccctccgcggctcatggcgacgacgcttggcacatccgggaccctcaggccgctgcagtcgcgtcgccggctgctcgggccccagccccggccgctgtggtgactccgccgcgcctcgccgtcgcccccgtcccgcccgccgccccagccgccagggacatgtctaacctcggaggccgtaggaacgggcctgtcgaagtgcgcctgacagggacatgccacccccgttttaggagaacttgtatttggtttgagaaggaattggaatcacggcgttatcctaccttcaccatttacccctgataaattgtccacgtttatatttggagtctcattctattgcccatgctggagtgcagtggcaacagcgtgcagtgagtgatcccggctctctgcagctttgacctcctggctcaagtgcttttcccaccttggcttcccaagtagctgggactacaggaatcatattgacaggtcatgaaaatgagaccaaaagatctctggaagcgattaatgataaaatttcatggagaagaaggccttgactacggaggggttgccagggtgatgacaactccgtgagggtggagattatacatctctcatcatttcagcaacaaggaaataaattagtggcagagtaagggtgacttgagatctcttggaagcgaggatcctttaaaaatcctggaatatacactgcagtaaaagaacaaagcatacctcagccttaaacgactgaagaagaatgtcaagtggggaagtggctttggttttcagtttgtgggttctgaatccacacaaagacaggattgctttctgaaaacctgaattaattattgtccttacctcaataagacaaaaaattagaatcaaaatcgttagtattacagtcacagatatcaccaagattagtttgttgttatagccatatcctggaacttctttcgtgagctaaaaaaaaaaaaaaagaaaaaaggaaagaacaattaaaaaaagagtgaaagctaacttttcaaaaccccagtattctagttgagtagcttacaggttcttttttttttttttttgagacagggtctcactctgtcacccaggctggagtacagtggtgtgatcacagttcactgcagactcgacctccctgggctcaggtgatcctcccacctcagcctctagagtagctggaattataggcacaagtcaccacacccaactaatttttgtattttttgtgaagatagggtttcactatattggtcaagctggtctcaaactcctctgacttcaagtgatctacccacctcggcctcccaaagtgctgggtttacaggcgtgagctaccatgcccagcctacagttcatcctgtgcactaatctgtttcactctctaaatgaacaaagtgggagatcactgtcatggccaaagttacatggccaagacaaactatggcctgggagtcccaggttctcctgtgtgggcactttcctggcatatgctaaatgatgggaaagtctgggtctcatgtttctgtgtggtcctcacctcactcgacttctgctctttctgttcactctgggcttctgtgctttcattaatatagttcttagttttccactggtctgtatcccattctgccttggacgcctttacttcctgctgctcactgagaagcctcatcaggaggcctgtcctggagatgagcttggcacaggtcacttgcacataggtctcggagcagtccgtcttcaaggtccagataacatgagaaatgaagcttctcacatcgttgttggggatgacggactgtagttgatgggttagcggaatttcaaactgatcacctggggatgagagcaatgggtaactgaagcttttgggctcgggggacaggtcagtgcccacgttgttgtattcccattttgtctcagtttgtttaacagttggctctaagttgaatgcagtcctagtgggaatctgcctcaggaagatgattgtagtttgtgttttcagagatggtgccttctggcatagtagtgttttccataaaaacgttttcttcagaggcatttcttgcagttgtgttttttacagtagtgttttctataaaacattctgaaggagcaaatacttctggaaaagggttttcttgaggactcaggtctcctgaggatgaaaaagccccttgtgaaggggaatttatgaggctcttcactgcagagaacggcggcctctttgcgagcatcagtctattgagataacttttctttctcaattttctaatctttttggccttgggtgttctgtgggccacacgggagtgagttttatgaaagtggtatttttttctggaatgtactactcgtttagcagccttcatattttttaactctagcctttgcattttctaaaattaaaatggtgtaggctaagtctttcaatctgtctatgacctgaggtagggcttttgcagttgtggaaggcagaaggcatgcccatggagaagggtttcagcaaagaggagactgttggcttctgctcttgggtgaatgaaggcttggtgtagacggcgtttcccgctacgttctcgggcccctgcactatgtgaggctattccagctcccttggggctggactcccgagccttttttctttggcagcattctccccaaatgcctgggcaccctgttccctcctgatactctgctttcccaccgctttgaagggccttttctggatgctccttgggcccatgaggactctgttcactctctgcaggtttttgcctacactttgaacctttgccaggctgttttcctgtggttggacagtttctaatttcttttcaaatatttggcatttaaatttgttacctagaaggctggactgcataagcatggcagttttttcttcccttttaacttcatcgatttttttcttgtatttctgcatctaacatcttttctagcaaatacacctttctcaatttattttcataagttgaattgttggatgcaggttgaaacaaggggctctttgtattgtttttcaaaacaggagcttatctccatcttgcacatttgaaaaaagcagtttaatgaatggtaacaacattgattccgcatctagatttcctgctgagaaataaggcaagatgtaatttagcgcagtgataacatcactcttatcataagtgtctagctgctcactcccaaagactgacaagttgatgccactgctgtctgaggctgcctccggctcagtagtcagctcaatgctcatgtgcttcttctgggcttgtaacaccttcatgaacactctttctggattccctacagatgcttcttcacctgtcaaaaaaaagagactgctttgatcatgaaagataatggaacggcatgcatcagtccacagctgtatgccccagtcacacaaagtaggagtcagcaaacattagagtgccattcagagaggagaaacacacacccaagcctaaatctatgaaatggcaacaacaaaaggaggaaaagacatcttttgaaaatatggctacttacttggaacattcctagtgtgacatagagtaactctgcttaggattatttcgttgatccccaggggccaattgcccagtgctcagtcaaagcccaaggtggaagacaagtgcttccctgatgagctgtcctctctgcagactgctccgtaccctgtgctgtcctgcctcagatgcagagagagcacaagcctccctcctcatcctcagtgtgcctgtgttcttactaccatcacagctgaatgcaatgaaaggctatcctctgagaggagcagggtggagatgctaaagtggaggccccctcccattgctgatagatcctcatctggcatgcactccacccacccaccccattctctgctaccacatatcgtagccccattaaagaagaagtgacatggaaaaaacactgtgcccaccctagtccttttcttaaatttgggcagagatccagggtataggttaagagatttttaatttgccagatggtatgcctatgttgttaaatatacaatgaatctatggtatgatagcagtttctggataaacattacttgaggtcctaaaatgcagaagggaaaaagcagcttttgtcagatgcttactttgctttcatttcatctctaatattttggatggggaaccatccaaagcttctggccgcatgaaggtcaagtgtgccagtgtgcagctgggtttcttttccagaattaaaagtattttgggtggtgctgagggtcagaggaagaagtaaaaattgtgagaaaggagaaacatgggcttggggagaacccagaattggggacagaagacctggcactaagctatagcacttagcacctctgatcttgtttttcctcgtccgtaaaaggagattaacaatgcttttctgcccacctcttggggagaagggaataatttagttggtaaaaaaaaaacttttgaataataagcaactctgtctttatataagtagccaagcattattaattatccaccccatatcactggtagatacctgtattcaagctatgtggacataaaagcagtcacattttagaagtcatgaagttggtgctaataaacctaatctacagaaaaactcttgaaagcacttgagcatttgttctgtgaatagaaaggtttgagattcagagcaagttcagagttggatggtctaagaatggaaaagccctccattccattagaagagccagggagcaatttctggttatggaaccagaagctctcaggcttcaaataaaacagcatcacttgtactcttataaaattgtaaaaacaaaaacaaaactggatctacatttgtcctaaaaggcagagagtacttgagacctcatgcatataaaaccagcttacaaactacatcgcactatatgaagaaattatcactgggggcaaatcaccaagtagggagcacaatacacagggtgtggatgttaatgtcattccctagccttctcattcccttctcttggtcctttatgcatatggaacagttccattattaaattttgtaataataactgagaacctgactcccagcaagggagtagttcagaagttgagggagtttaaatctgaatgagtaaataaagcaattatatcattagcttaaaattttatcatcaattaaaaataaatttaaaaacaaatacttaaaataatgtaacaatttatcaccaggcaatttggactcacgacaatgtgtggtgtttgtcagacatgcactgttgcaatgcagcttgactgtcttgcagacagcctcaatgctatttttaaattggcagaggcagcaggccatatggctaggtaagatcctataagatgaaaacacaaaacaataaatagtggtaaagcagttacttgagtaggcaaaggaggcaggccaacgctaccacagggctgggcagaaaggtgttactgaggcctgtggactggacagttgggtaggaaccagaagggcaatgggaaggaggacaaaggtgcccaactgaagggtaagcatggcagtgagtatggtatgcctagaataaaagtggttgggattagaattgggtgacactgatcagtagtttaattcagaagtatctcttcccaactcaaaagtctcactttgggctgaaagtacacaggaagaaagtagacttctaagaagagtctgaataagcccccaacttctggagtccctttctcaatttctgttggcagtgggaaatattagaaattactctgggcattaaaaatagctcagtttaacctggattgtggggttaaaaaaataacagattgcattggccaaatctcgacaatttgagcattcaaaagagtaacaataagctacaacataattaatatataaaaaagaatccatgaagaatgatatttaaaaagagagggagttgttctttaatgaaataatgccagctaatgaatgtagaaggaatgacagaattttaaaaagtgttactttgcaaccaccaatgtaataaaaattgattcagacaaggattgtcattgatggacacagctgggtgagaagacatttgagaacaggatctttactgatctcaaagtaccaccacacagatattttattaattaccaaagggaaaaattattttttatttttttatgagacaaagtctcactctgtcacccaggctgaaatacagtggcaaaatcgtagctcacaacagcctcgacccccaccctgggctcaagtgagtctccaaattcagccccccaagtatatgggactatagtcttgtaccaccacacccagctaatttttttttttttttttttttttttttgtatttttagtagagacagggtttcaccttgttgctcaggctggtctaaaactcctggactcaagcgatctgcccgtctcggccttccaaagtgctgggattataagcatgagccaccacacccagcaaaataactacaatggagagatctggaagatcaccttaaacaagtgatcaaacttagcattatgagccacctgcaatcacgaggcaggaaggatatatcacctatgcagtatttttcccaaaaatgcttaccttgaatctcatcatgtggaaacagacaaatccagattatgggacaatttacaaaacaactatctttgactcttaaaaaatgccatgtcatgaaagatcaaaaaaagtagaggaatgttttagattaaaggaaatgaagacatgacatgcagtgcctaatctttgattggattctgtgctattctttcatctttctggtatgtttgaattttttttgaaatataaacttgggcaaaagagataaccaagataattgattaatttattgttatggctcattgggggcagtttcagagaaataaaaacaatctttgtaactggagtaaattctcaaggttaatcttaagcaatatagcatggtcattaagaatacagattccacagccagactaggcttaaatcccagctctgtgaataatgtgaatttgggcacattgattaatctctgttccttggccttgtcattataatagtacctacctctaatgagttttgaggattagatgaatcagtacctgaaaaatgcctgacacacagtgagtgctcaataaatgttaactactgtaattattacattacagaggttgtgggggggccttttctgagtcctccaaaaggatgactttattagggccacattaagactgtggaaacagaagagtattcaacagataaatgaagtctatgagttgggggtacaatttatggagttttagattaaaactgcatccaataagttggtctgacacatctttcaaacctatagaggaataatcacaagtgactagtattcctttgggtccaatagaggtctctgatcttcatacagattgaatgtacccagaaccgtaacctagcattttacttatatagcaaccttaactctgacacaaaagtgtttcttttgttttgaggccaagtctcactctgttacacaggctgagtgcagtggtgcaatctcacttcactgcagcctctgcctcctgggctcaaatgatcctcccacctcagcctcctgagtagctagaactacaggtgagtgccaccatgcctggctcatttttgtattttttgtagagatggggtttcaccatgttgcccaggctggtctcaaactcctgagctcaagcaaccctctctccttggcctcccaaagtgctgggattacaggcatgagcctgaagtttaattgttattcttttcctgcccccgacttttgattttaaacattttctttttttcctttaagccttaggatggctaggaagcattttcagatggtataatgaacacctgtataactttcatctggaatcaacagttgctaatactttgccacatttgctttccatgtgtgtatgtctatacattttctggacaaaccatttgagagtcagttgcagacaaaatgaccctttgccattaaagacttcagtgtgcagcccctaagaagcaaggcattctcctgcataaccagaggactatcatcacccaatggaacttcatattatcattgtctactatgcagcccatatacacattttcacaattgtcccaatcataacatggcttaaaaaattcagaatccaatccaacatcagacattgcatttagcgttcatgatttttttttttttttttgtagaaagacctttaatacttctgtttacaaaattcaggcatacatttcagtttgccctgaaccgtgcccaaggctgtgtgctcatctctgccaggatgtctgtgccctcatgtactgctgacgtggagggtgcagagcagagcctggggtctggaggcttcgctgggcctcagggggaggggaatgtgaatgtggcccggcccagaggaccctccatttcatcagttttggattgggcaacagaggaagcagatgtctgggctgcagacctgctggtctgggcaggggtttgggaaccccggggttgcctgccatggtctagaggagatggctgcggctacttcccacagggtggggtggccactaggggcttgaagatttgctcactggagcctttgtgcttggccctcagcagcacagctggggctgtggtcccgtgcaaggtgtgctgagggttgtgtaggtggctggtggtggcagcttgtgccagagtgacacaggcttctgtgggcctggctgggggcagttaaaaagccgaaaaggtacttggctttgtgagggcaaggcttgggaggcggggccctgcaggacgtcatgttctctgtcctggttaggaggggggaggtgttgggggggggaaggtggggcagatcccaactggcccctgtccctgggctgcaagctgacagcaggccagggaagcagtgaggccttctgccctggccttggggagaaggagtgcatgtgtgaggctgggatacagtggccctggagtggggctagggctcatcgccagcaccaggcgcggggaggatttaaaaccagagtcctgtctctgagccaccccacagggacgcttgatccaagcctgttaaggccgtggaactcgagggtaggcagtgggctgggccaccgtgggttcaggatggaggtgtgggggctgccctggaggaacgttagtgtttctgagagcaccttagtgttgctctgcctcagccagctgggcaaggagttggggagcggggaggaggccggcgccatggagtggggaagggagccgtcactgttggggatcagccgctagtgtaaacgagcatctccacccacccagataagccccccacccaagtgtggagccctgctcagggagtacctcaagggccccggcctgggtggcgccagcaggcttggacactgccctggcagcaggggccccgtggaaggcaggatacccagcagggggtgccacccagtgtggatggaggggcagggcgggggcgcccctgagtcactgggcagaggcacgagcacgtccacatagctgagcaggaagaagcccgactggccgtccagcatgccctcgtatcagttctcgttcatctggttgatcagcatgaagacatcgccctcatggaagcccagctccctatctttctccggctccaagtcatacagcccttgcagctcggctggtctaggggcggcatgctcctgctaggagtccagatggacttgtcgtaagatcggaaagaggacgaagctgcgatcttggggactgtggtgcaggggaagccccggttggactgctcaggctttccgaggtcaaagcactcccggggcttgggcttatactcccgcctgggtcgtgaggaagcttcccggatcctgcacttgaacttctgggccagctggtccaggatctgcacggcctgccggtggtagtccagctgcgcctctgccaaggccgggagttgactcacctactggatgtcggtctccaggaggttgtgcatgctggtttctgccacctccttggactcctcaaacttctccagcgcctggtgcagctcctcttcggggatcttgccctgccgcttcttttagtcaaagtccaggcggcggccctccagcttctgcaggtggtgctggatctccttcaggtctttctcacataggttctggagggggtcaatgaagttatgcttgacctctatgtccagggagtccttcacctctgccaggtgcttcatgggctcgccggcatccagcagtgcatcgctgaagttggactcgtcgcgcagctccttcccatggcggatcaggcactcgcccaggagcccccctgactgcaggtagccggggttcttcacctggccccggatcttgcacgcggtgttgagcatggtcagtttagcctgcgaggctgggttgggctgcgggtactcgatggtcctggacagtacttacgtcaccgccttgctgatgacatccaccttctccatctcttcgaagtcatcatccagcttggtcccctcagcccctccgaccttctcactgaccagttggctcgccttgtagaactgcttcttcagcccggcaaccgacatgctgcctcctgccgcggaaccttccgcccagacccccctgcccagaccccgccagcgacaggctcccgggcaccaccaaccctctgcgcgcctcagcggcccccatcagcaccgcctccaccacccgcttgccagctcggcgcccgccccggcgccgcctcgagttcatgattctttagtctccttcaatctagatctgttcccaggattgtttaaaagcatactgacaaacctttgagactgtaaatgacctgaggtatacattatctttacttgagaatagtaatttttcaatatgcattaaagatcattacacatgagctaaaaaaaaaaaaaaaaaaa

SEQ ID NO: 43 - DNA, 1752 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcagccaagcttcttgaaaggcctgtctacacttgctgtcttccttcctcacctccaatttcctcttcaacccactgcttcctgactcgctctactccgtggaagcacgctcacaaagggctaatctcgggccttgtcgaaggaagaggctgcagacgttaatgaggttagctgctggattccagtattcgtcgcataaggatccttctttgtctgcgaaggaaaaacacactgattatcataatgaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaaaaaaaaaaaaaaaaaaa

SEQ ID NO: 44 - DNA, 1785 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcagggctaatctcgggccttgtcgaaggaagaggctgcagacgttaatgaggttagctgctggattccagtattcgtcgcataaggatccttctttgtctgcgaaggaaaaacacactgattatcataatgagcaggttcccagtgcccccgacaagcccctgctggtgtctccatctcctgccaagcatcctccagtgcctcctcctgtgggcctggcctcagggctatggacagactcctgtcccatcccagagacccctcgtgatcgtgccctggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaaaaaaaaaaaaaaaaaaaaaa

SEQ ID NO: 45 - DNA, 1653 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcaggtaggggctttgcagagaccatcgacgggctgacttgaccaagcttcttgaaaggcctgtctacacttgctgtcttccttcctcacctccaatttcctcttcaacccactgcttcctgactcgctctactccgtggaagcacgctcacaaaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaaaaaaaaaaaa

SEQ ID NO: 46 - DNA, 9701 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcagccaagcttcttgaaaggcctgtctacacttgctgtcttccttcctcacctccaatttcctcttcaacccactgcttcctgactcgctctactccgtggaagcacgctcacaaaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatcggcagactaggatggaagtgccctgtgagctggggggcccttcaaagggccaaggagaaaacgcaggccgagggaccagccttccaaatgggcttcaagctccaatgacctccgctcgccccctcgaaatgtctggaaaacataatgggcagattttctgtcttcaaagtttccggctaaacctcttcaagttctttattgtttgggactgagacactcagccatgttaatgggtagtttcttttgtatttgccttgaaaggccaaaatatttttatattgccacagacaaagccacctatttaaaaatgaactccatgtccgtcgtttcccaccaggagactatgtaccatgtgtgtgtctctatgtattctggggtcttgaaacaggtttctcatggggatggtcattcaccacggtccagaggggcagaacaggcggcgcttgccttgcccagggggcctggggaacgtgggccctcatctcagatctgcccccagtatgtttaggacgcgagccccagaaggatctgggagtaaacttaacattcactgtgtctctgctctgcatccgccatttgtgtgtgtttctggactgtgggctgtgtgtaccttggttggtgactcagtgagaagaagcaggaatgccaaagatactgtgaatgttctgagttttgttgctgttgttgttgagaggttgtttcactggtatctattgcattgtataataaatgaccagatgaatgaatgagtgaagcaagagagaatgaataaacaagtaaataggtaaagaagtaagcaagccaggatgagagtgtgtgtacacaagaccatggttcatccgctttgatggctaggcaatcaatatataaatagaaaaaaaccagtgaatcactaagtaatagggcaacacacaaagcgatatcaggtgattatggactaaggggtatgtgtaactcaaatatatgcctctgacatttgacaatgaaaaagaacctaaatgaaagaaagaatggatgtatgagtagtgaagtgcagaatgagacatagattttgaggcccgtcaaaatgaaaagatgcaagttagggaacaagtgatcaaaagggagaagggaaaggttttttttaaaaaaccaaaacaacaaagaaaggttaaaaaaaaaaacagactagaggatgagtaatgagtaactctgtaaggaggaccatgtcagactattgtaagctaagcattaggactgatacaaataatatatgctcctggcatagaaaaataaaccacagagaacgagttcaaagaatagcaaagaaagaaagaggacccagtgggcgaaagatgagagtgtacttttaccaaaagttatctaagcctgagcacttgaagtctgcacataaataaataaatgacaaaagaaagaaaaaaaggccaaaaagtctacattgcgtgtgtggatggatgaatgagcagtgggagtgcagcgccaggtgacaagatgttgtgaggggttttgagtcatccagtcctgggcactgaggtctgttagatgaaaggatatgagaaaggtaatattggtaaataaagaaataggaaacaatgtaacaaatgttaagtacagaaatacattaatgggtggtaaataaagatgtaaaagaaggcaatgcgatcgatggtggcaaaagatcatcacagattaagggctatggctggtccacttctagaaaaccacaggctgtccattaaataatgaacatctaagtgaacaagtcagtgagtacctaaatagacaaggatgaggtgaatgagaagacatggccccatgggtcctcctgatgagggtgttggggtcccccctgggcaccccagctgcatgaaaatgaaggacaggaggtatggaaagctatgacagaagagagaaaggaacggtaaaaagaaataacaaccaaatggataaatgggtagatccacgagaagagttaggctaggacttgtcataagggcacctgactccactaatagaggaataaatgcctaataaaaagagagcaagcaggaaggaaggatgctatgaatgcaggaaggaagtaatgagtgagacgtggaaccgcacggccaaggatggacgtttgcgggtggctttttgatgcgtacagccaagccactccatggcaatgagctccgaagacaaagtgcaagagagaatgagtgagagagtgagagagagagaaacaataaaaaatgggaagaaatgtaaaaaggaagaaaggaagagaggtaatatattaaggaataaatacatgcatgcagatttaagacagagccatgctagaacaggaatgaaaggctgtgtgaaccaagcagaccgcttaattggcaccagtgctgctggtatggtcaatcacctactcaactaaggaacggctcaaagcatacacatgggagggaggagtggggccacagagagagggcccattagttgcagattacgatgtatccagttaggtgcacctgccttcgagaagtgtaaaaataagtatttacatagaaagaaagactgaatggatgcacggtgaatgcatgaatgattgaacgacagaaaagatttgcattgaccgatgaggagggcattgtagacagggatgagggtcattgatcctgggtgcagatctccaaaagaatgacagaaagaaagagggagtggtggaaagaaacaataggatgggaaaaaatgaaaatagaaaaaaggaagtgaaagagataataaataattagatcaaataagttgatgaaaggggactggtttagcacaagccatccacattaattcaaacctgtggctctgaagtttgttttttaaatgaccacaagtgtaagactgaatgaaagaataaatgcgtgcattccataggatgcaagaaaaggagtgaggaatgggaaaattggaagaacgagagagggagagatgtaagaaaagaaaggaaaagtgaagtaggcatatgaaagaaaaggcacttcttggacaagcactgaaatataatgagacagttttacccattaaatataataaacagtaaacgttgaggttcatcaataaaagcacagatacctgaatagaggagtgacctgaatagaattcgttcagccgaacgaatgagaatggatgattttcactatcctgtgcactcaaggcccaaaagagaaagcaagagaggagagaatatggaaacgtatgacaggatgtatataagcaatacaaacatattgaatgaataaataaagacataaatatgtgggagagtggaccacgcaaggacaaaaagaggagagaaggcagcaagaattatgactaattcaaaactgggttcctgagatagttaaataaatcctgcaccaaatccccagggggagaaattaacaaacaaaagacagccccacacggaccagtgtgcagaaggctccaggaaccgcagattatggttaatccaattctgtgcacctgaggtccataaataaaagaataagtattgaaatgaaagaatgacagaaagaatgaatggacacatgaacgactgaattagaaatggaaatgcctggcacagccaggaaggagctgcccatgggattgtcattcatctcactctgggcacctgaggtccataagcgtgaaaagaggcaggaagagaagtgtcagggagtcaaagatagagctaaggaaaggcaaaaatgaaactaaatgaaagcgaaagggaaaataaagaaaaaccaataaaaaagagaacgaatacgtgggtgtatctgtaagagtaggatctgttaggattagtcataagactgtcagtaatcctgaagatggatgagataatccaggcccaggttcccagggggagggaaaatggagaaaatataaaaagatgtgaaaaaggaaaaaggaaaggtaataaacaaacaaccaaagtgataaatggatagttaagggaggttgtctgaacagggattataattagtttacatacatactccttaaacagataaatacattacacctttcaaagaataaatgaaaaatagagagacatacctggctccaaaacaaggctgtatcttctgccactgtaataaaatagatgcaattgaggttcataaataaaagaataaatacttaaacgtgaaaggtgactaaatgcggggaagaaagattgcaaataaatacatgggccaaagatgtttggtttgcccatggagttttaattaaaaaaattaataaggaaaacaaatacccaaaataaggaagactgacaaatgagtgagtggatgagagagtgaatggtgcttgacgtaggagcagtagtgctttagggaccagcatgaaggtggtgaccgggagccctgattcatgggattctgtccacctgactttataagaaccaagaatggctgggaatggtggctcacgcctgtaatcccagcactttgggaggccgaggtgggcggatcatgaggtcaggagtttgagaccagccagtttgagatcagcctggccaacatggtgaaattccatctctactaaaaaaatacaaaaattatgggcgtggtggcacctgtctgtaatcctagctactcgggaggctgagagaggagaattgcttgaacccgggaggtggaggttgtagtgagccaagattgcaccactgcactccagcctgggctacagagcaagacactatcttagatcaaaaaagaaaaaaaaaaaaagaaggagaaagaaccagagaaacataaggaagagtgagaggaagaaagaaagatgcaatttgggaagaaatgaaaaagaaatgaataaagaataaaataatgtaacggtcaataaataggacttgtgaatggaggcctttaggccaaaggctatgattaatttcaagctatgttactgaagtccataaacaaaggactcagatctaaatggatgaacgaatgactggaagaaagggtggtaggaaggtaggaagaaaggaaggagggaaaaagggaagagaggaaggaaccttctttccagtcctgtgttctagacagtggaatgaagtggtccccagggagggtggctgtaggcatgtcatgtgcttgtcacatgcacttgccctggcagggaggagctggctcaggaagaccctggtcttggggtgctgttgccctatcttggctgtgtgggccatttcactgcatctgtctcttcctcagtttccccatctgtaaacctggagtggcaccagctgcctactagagttgatcttatgtgtctctgttgatggtaccccatctatggcctggataggcaggaagggcttggaccctgagccccgcagaaggttgcatgaacgagtggtgtgaagcctgttgggtagcttggccactcccgcggcatgggtcacctgcacaggaggttttgcccaccagggggcagcagagggtcagggagcaataggccctgggtggagcatgggccccgcctgctgtgtgccaccctgggtgtggcacctactcacatccaggggttggtgcagggaaaggccagaaggtggccaggcgcacctgagaagggggacccagaagccccgggacccaggagccctgggcaagccaccagaaaccttgttcttgcaactctctgcagtgtgcccaggccaccctctggcctggtcttccatggggcagggcgcccacccttctcaactcaggtttccctgggcagcaggtgcacctcagcacccctggggttgcagaagtggtccggggaccctggcttccttgacatgccatccccagagcctggttcaaggcctctctgtcttctcggctgtttcacgacgtgttttgtaacttggcgggattgcgtttcgctgtgtcgaggttgtctcttctctgactcgccctccgggggactgccggggtaaatctggagagttgctcgtgctgacagtcctcccccagggcctccccggttctgttgagtctcctttctctgtagtggaggaaatgtgtgtagttttgtgttgtgtgcctgtgtttgtctgtaaaagcaaggaccaaagtctcccttgttgacctctcaattcctatttgggacatataaaaacactggattcttaacaagcgcccggagcagtaggagcacagcttggatggactcaggacttgtggcagggagcacgtgggaggcaggggagtggggtggggccaggccatctggagtgggaggcgtcatgctcagagtgactctgtagacgctgggtgggatggggagtgcgggcgcaggcatggatggggctgttagctagtgtgatgcttgaggtctgagctgatggcagcaaagtggggtgctcaggaatcaaagctatggggttatagacaggatatgaaggagggagggaggcaagaagaagggggtggttcccacgcttctagctccggccgagtggatggcaacagcatttggaaggcggaggacatggaattcatgtgtcaggagccaccttccgagcctccagtaccacgtgtcagggccacatgagctgggcctcgtgggcctgatgtggtgctggggcctcaggggtctgctcttcttctctttcagaatctggggctccaggctatgccttggctggactgaggtctgggggtgcacttattatccctggggacacctgctgaagcttctccctgacaagctgtgtcactgttggatgaggatggggcgggaggggttcagggcagaagaagaccgggagggtctttcaaaagaactcatgtacggctgttaaaaaaagtcagcagaggctcaggaagacttaaagtgtgcagaaggcggggaagggagggcccattgcatgcaccaagaggaaattggaaggaacaagcgacgttggctgctaggagagcctgctcccaacatctaggggctgtcctgacgggtcacagtgggtcgaactgagccaatgagagcagctctggggagacccactggtgccctggaggctgggtgggtttgggttggatgaattctgtgtgtccttttggaaatgtggaggccatgaggggggatcagggctcttagggttttgacccttaagagttttgtatctgtaattcaaaggttctttagttctgggatgctgagattcgggatagggttcctaatggcacaaaagccagagataaaacatccttcacgtgctccctacccggttctttctgtaccagacccacaaggtccgagttgggatcctagtgctcctgtctggtcagggcctatctttatgtgttcgttaaacttttaacaatgagaattaattctgtctcttgacattgtcatttgcatgctccccacacacaaatcctttcctggtgacaccaggagctacaactctccttggcctcctcttgtgactcccaactccctccttgggaagcttggcctcaggacctctgggatagacaggccacgaatcctgctgtgtcccgttgtgttcctaatataaatggtgtggatggcacttgacctagagcagtgggaaatgcatgcaccactcaacattctgacatgtcacccattttacattcttacaggcatactttttttaaaaaaagagtgtctattctttaatgagcatcccttctttaaaaaaacctaattgccattattcaccacatacacttttttttttttgtatcctgcctcttctatttaattttctgtcatcaacattttcccttgttccatgaatcttcataacctcacttgctgcgttgtgccttgttgagtggctatggcatcattcacagaaccattctgttattcttatgtataaccaccttttaaaaatattatgaataatgccacaactaactgcttaaaacaccctttttttcattcttaagaattatgttcttccacccagaaattatcattgcttcactacagatcagtttcccctgctagactgtgagccccataagggcaaggagcttattgaattggcctttgtatctctgatgcccaacatgttgtagactataaataaatgatgaatgagtggatggaagaatggaggaaggagcgagtgagtgagtgtttggctgatggataagagggtggaaggataggcggaaggatggattggtgaatgaatgaatgaatttcctttggttaagtctcttgaaagaaaggctatggatctttgtatggatgttgaataatttcagtaagcttacagcattttacaatgttcagcaatgtatgaccacttaattaagatatggctagtttgtctctgttataaagtacttttgcattactttaacttgcattgctttaattactaatgatgggtgaacactttgacctatgtttgttaacaaattgtattttatcttctgtgaaaaaaaaaaaaaa

SEQ ID NO: 47 - DNA, 1731 bp, Homo sapiens
gcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcagccaagcttcttgaaaggcctgtctacacttgctgtcttccttcctcacctccaatttcctcttcaacccactgcttcctgactcgctctactccgtggaagcacgctcacaaaggttcccagtgcccccgacaagcccctgctggtgtctccatctcctgccaagcatcctccagtgcctcctcctgtgggcctggcctcagggctatggacagactcctgtcccatcccagagacccctcgtgatcgtgccctggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaaaaaaaaaaa

SEQ ID NO: 48 - DNA, 1687 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcaggtaggggctttgcagagaccatcgacgggctgacttgaggctaatctcgggccttgtcgaaggaagaggctgcagacgttaatgaggttagctgctggattccagtattcgtcgcataaggatccttctttgtctgcgaaggaaaaacacactgattatcataatgaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaaaaaaaaaaaaaaaaaaaaaaaaaaaaaa

SEQ ID NO: 49 - DNA, 1547 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcaggtaggggctttgcagagaccatcgacgggctgacttgagcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaaaaaaaaaaaaaaaaaaaa

SEQ ID NO: 50 - DNA, 1656 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcagcaggttcccagtgcccccgacaagcccctgctggtgtctccatctcctgccaagcatcctccagtgcctcctcctgtgggcctggcctcagggctatggacagactcctgtcccatcccagagacccctcgtgatcgtgccctggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaaaaaaaaaaaaaaaaaaaaaaa

SEQ ID NO: 51 - DNA, 1793 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcaggtaggggctttgcagagaccatcgacgggctgacttgaccaagcttcttgaaaggcctgtctacacttgctgtcttccttcctcacctccaatttcctcttcaacccactgcttcctgactcgctctactccgtggaagcacgctcacaaagggctaatctcgggccttgtcgaaggaagaggctgcagacgttaatgaggttagctgctggattccagtattcgtcgcataaggatccttctttgtctgcgaaggaaaaacacactgattatcataatgaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaaaaaaaaaaaaaaaaaaaaaa

SEQ ID NO: 52 - DNA, 1506 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaaaaaaaaaaaaaaaaa

SEQ ID NO: 53 - DNA, 1687 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcagcaggttcccagtgcccccgacaagcccctgctggtgtctccatctcctgccaagcatcctccagtgcctcctcctgtgggcctggcctcagggctatggacagactcctgtcccatcccagagacccctcgtgatcgtgccctgtagtcaccccactccaccgaaaagtaaactgcaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaaaaaaaaaaaaaaaaaaaa

SEQ ID NO: 54 - DNA, 1621 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcagggctaatctcgggccttgtcgaaggaagaggctgcagacgttaatgaggttagctgctggattccagtattcgtcgcataaggatccttctttgtctgcgaaggaaaaacacactgattatcataatgaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaa

SEQ ID NO: 55 - DNA, 1714 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcaggacgaataatttatggtcagtgaaaatccaggccccagtgagattcgagtgggctgtaaaatcgagagtcctgctcccacaggttcccagtgcccccgacaagcccctgctggtgtctccatctcctgccaagcatcctccagtgcctcctcctgtgggcctggcctcagggctatggacagactcctgtcccatcccagagacccctcgtgatcgtgccctggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaa

SEQ ID NO: 56 - DNA, 1735 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcagccaagcttcttgaaaggcctgtctacacttgctgtcttccttcctcacctccaatttcctcttcaacccactgcttcctgactcgctctactccgtggaagcacgctcacaaagggctaatctcgggccttgtcgaaggaagaggctgcagacgttaatgaggttagctgctggattccagtattcgtcgcataaggatccttctttgtctgcgaaggaaaaacacactgattatcataatgaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaa

SEQ ID NO: 57 - DNA, 1855 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcagccaagcttcttgaaaggcctgtctacacttgctgtcttccttcctcacctccaatttcctcttcaacccactgcttcctgactcgctctactccgtggaagcacgctcacaaagggctaatctcgggccttgtcgaaggaagaggctgcagacgttaatgaggttagctgctggattccagtattcgtcgcataaggatccttctttgtctgcgaaggaaaaacacactgattatcataatgagttcctgacctggccatcccggggtgcccttgaccagccccgtgtctcctcagggtgtcccagcaccagcctggcacagagtggggctcagttagagtatgtgggatgttggtttcgccaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaaaaaaaaaaaa

SEQ ID NO: 58 - DNA, 1595 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcagccaagcttcttgaaaggcctgtctacacttgctgtcttccttcctcacctccaatttcctcttcaacccactgcttcctgactcgctctactccgtggaagcacgctcacaaaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaa

SEQ ID NO: 59 - DNA, 231 bp, Homo sapiens
gcggcccgggaatctacgtcacccgaaaagcgactataaacgccggcgcctccgtccccagccgcggctcgggaatccacccgaagagtggctataaacgtccgcgcctccattgcgctctcctcttcacttaggacactggtcctcccacgcctgacaccgacgtcgccaggaccgcggggttgggggaacttggctgtcccacgtctttcaaataaagctgttttgtct

(19) United States
(12) Patent Application Publication
(10) Pub. No.: US 2018/0023080 A1
(43) Pub. Date: Jan. 25, 2018

(54) Long Non-Coding RNA For The Treatment Of Endothelial Dysfunction

(71) Applicant: Johann Wolfgang Goethe-Universität Frankfurt am Main, Frankfurt am Main (DE)

(72) Inventors: Stefanie DIMMELER, Frankfurt (DE); Reinier BOON, Frankfurt (DE); Nicolas JAÉ, Hanau (DE); Katharina MICHALIK, Frankfurt am Main (DE)

(21) Appl. No.: 15/548,329

(22) PCT Filed: Feb. 3, 2016
(86) PCT No.: PCT/EP2016/052246

§ 371 Date: Aug. 2, 2017

(30) Foreign Application Priority Data
Feb. 3, 2015 (EP) 15153717.2

(51) Int. Cl. C12N 15/113; A61K 31/713; A61K 31/7105

(52) U.S. Cl. CPC C12N 15/113; A61K 31/7105; A61K 31/713; C12N 2310/113; C12N 2310/14; C12N 2310/341

(57) ABSTRACT

The present invention associates multiplelong non-coding RNA (lncRNA) with key functions of endothelial cells. The lncRNA of the present invention are therefore useful as novel drug targets for the manufacturing of medicines for the treatment of cardiovascular diseases or pathological angiogenesis in context of proliferative diseases such as cancer. Modulation of the function or expression of the lncRNA of invention can induce or repress angiogenesis and vessel growth or repair in endothelial cells. Provided are further methods for the modulation of endothelial cell functions in vitro, for example, in the context of tissue engineering.

### FIELD OF THE INVENTION

The present invention associates multiple long non-coding RNA (lncRNA) with key functions of endothelial cells. The lncRNA of the present invention are therefore useful as novel drug targets for the manufacturing of medicines for the treatment of cardiovascular diseases or pathological angiogenesis in context of proliferative diseases such as cancer. Modulation of the function or expression of the lncRNA of invention can induce or repress angiogenesis and vessel growth or repair in endothelial cells. Provided are further methods for the modulation of endothelial cell functions in vitro, for example, in the context of tissue engineering.

### DESCRIPTION

RNA sequencing revealed that the majority of the genome is transcribed, however, most transcripts do not encode for proteins. According to their size, these so called “non-coding RNAs” are divided in small non-coding RNAs (<200 nucleotides) and long non-coding RNAs (lncRNAs; >200 nt) such as natural antisense transcripts (NATs), long intergenic non-coding RNAs (lincRNAs) and circular RNAs. Whereas the function and mechanism of distinct non-coding RNA species is well understood and clearly defined (e.g. miRNAs), lncRNAs exhibit various molecular functions, for example by acting as scaffold or guide for proteins/RNAs or as molecular sponges. Therefore, lncRNAs can interfere with gene expression and signaling pathways at various stages. Specifically, lncRNAs were shown to recruit chromatin modifying enzymes, to act as decoys for RNA and protein binding partners, and to modulate splicing and mRNA degradation. Whereas microRNAs are well established regulators of endothelial cell function, vessel growth and remodeling, the regulation and function of lncRNAs in the endothelium is poorly understood.

Long ncRNAs vary in length from several hundred bases to tens of kilobases and may be located separate from protein coding genes (long intergenic ncRNAs or lincRNAs), or reside near or within protein coding genes (Guttman et al. (2009) Nature 458:223-227; Katayama et al. (2005) Science 309:1564-1566). Recent evidence indicates that active enhancer elements may also be transcribed as lncRNAs (Kim et al. (2010) Nature 465:182-187; De Santa et al. (2010) PLoS Biol. 8:e1000384).

Several lncRNAs have been implicated in transcriptional regulation. For example, in the CCND1 (encoding cyclin D1) promoter, an ncRNA transcribed 2 kb upstream of CCND1 is induced by ionizing radiation and regulates transcription of CCND1 in cis by forming a ribo-nucleoprotein repressor complex (Wang et al. (2008) Nature 454:126-130). This ncRNA binds to and allosterically activates the RNA-binding protein TLS (translated in liposarcoma), which inhibits histone acetyltransferases, resulting in repression of CCND1 transcription. Another example is the antisense ncRNA CDKN2B-AS1 (also known as p15AS or ANRIL), which overlaps the p15 coding sequence. Expression of CDKN2B-AS is increased in human leukemias and inversely correlated with p15 expression (Pasmant et al. (2007) Cancer Res. 67:3963-3969; Yu et al. (2008) Nature 451:202-206). CDKN2B-AS1 can transcriptionally silence p15 directly as well as through induction of heterochromatin formation. Many well-studied lncRNAs, such as those involved in dosage compensation and imprinting, regulate gene expression in cis (Lee (2009) Genes Dev. 23:1831-1842). Other lncRNAs, such as HOTAIR and linc-p21 regulate the activity of distantly located genes in trans (Rinn et al. (2007) Cell 129:1311-1323; Gupta et al. (2010) Nature 464:1071-1076; and Huarte et al. (2010) Cell 142:409-419).

Laminar shear stress provides a very important atheroprotective force by modulating gene expression of endothelial cells. For example, the exposure of endothelial cells to laminar shear stress but not to oscillating shear stress reduces inflammatory activation and prevents induction of apoptosis. Like shear stress, a reduction of oxygen availability—hypoxia—critically influences endothelial cell function and additionally induces the angiogenic response which represents an essential mechanism upon ischemic injury or during wound healing. The regulation of angiogenic activity in endothelial cells is therefore a key event in the context of several diseases. After the discovery of vascular endothelial growth factor (VEGF) many studies focused on the control of angiogenesis by growth factors, however, the control of endothelial cell functions by regulatory RNAs or epigenetic mechanisms remained unknown.

A critical role for lncRNA in endothelial cells is known. Repression of MALAT1 inhibited proliferation of endothelial cells and reduced neonatal retina vascularization and pharmacological inhibition of MALAT1 by GapmeRs reduced blood flow recovery and capillary density after hindlimb ischemia (Michalik et al. Circ Res. 2014 Apr. 25; 114(9):1389-97).

ANRIL is a long non-coding RNA which is transcribed from the INK/ARF locus and was shown to be expressed in tissues and cell types that are affected by atherosclerosis such as primary coronary smooth muscle cells, vascular endothelial cells, and human monocytederived macrophage cells. ANRIL transcripts were directly correlated with the severity of atherosclerosis (Holdt, et al. Arterioscler. Thromb. Vasc. Biol. 2010, 30, 620-627).

The lnc-Ang362 has been shown to be co expressed with to microRNAs (miRs) that are correlated with cardiovascular events. In particular, these miRs play a critical role in smooth muscle cell proliferation and neointimal hyperplasia in response to vascular injury. Interestingly, knockdown of Lnc-Ang362 reduces the expression of these miRNAs as well as cell growth (Davis, B. N et al. J. Biol. Chem. 2009, 284, 3728-3738).

In view of the state of the art it was therefore an object of the present invention to provide novel options for the treatment of diseases that are associated with endothelial dysfunction such as angiogenesis or cardiovascular disorders. The present invention seeks to provide new drug targets for these diseases based on comprehensive deep sequencing approaches of the endothelial transcriptome in response to shear stress or hypoxia.

The above problem is solved in a first aspect by a compound for use in the treatment of a disease associated with endothelial cell dysfunction, wherein the compound is an agonist or is an antagonist of a long non-coding RNA (lncRNA) selected from the group consisting of lnc-KPNA2, lnc-SERTAD2, lnc-Hyp_up_1, lnc-Hyp_down_1, lnc-Meg3, lnc-RNF151, lnc-Hyp_up_3, lnc-Hyp_up_5, and lnc-GDF7. For a better understanding of the invention, a more detailed discussion is provided below regarding the identified lncRNAs and their diagnostic and therapeutic uses for diseases associated with endothelial cell dysfunction, and regenerative medicine.

In the context of the present invention the above mentioned lncRNA identified herein are defined by containing the following exon/intron structure. Some lncRNA are known to comprise more than one splice variant. In the following, if for example referring to the lnc-Hyp_up_1, all its splice variants as explained herein below shall be included.

lncHyp_down_1 comprises the following exon/introns: the sequences as shown in SEQ ID NO: 1, 2 and 3, preferably in 5′ to 3′ order: SEQ ID NO: 1, 2, 3.

lncHyp_up_1 comprises the following exons/instrons:
the sequences as shown in SEQ ID NO: 4 to 6, preferably in 5′ to 3′ order: SEQ ID NO: 4, 5, 6; or
the sequences as shown in SEQ ID NO: 7 to 9, preferably in 5′ to 3′ order: SEQ ID NO: 7, 8, 9;or
the sequences as shown in SEQ ID NO: 10 to 16, preferably in 5′ to 3′ order: SEQ ID NO: 10, 11, 12, 13, 14, 15, 16; or
the sequences as shown in SEQ ID NO: 17 to 19, preferably in 5′ to 3′ order: SEQ ID NO: 17, 18, 19.

lncHyp_up_3 comprises the following exons/instrons:
the sequences as shown in SEQ ID NO: 20 to 22, preferably in 5′ to 3′ order: SEQ ID NO: 20, 21, 22; or
the sequences as shown in SEQ ID NO: 23 to 27, preferably in 5′ to 3′ order: SEQ ID NO: 23, 24, 25, 26, 27.

lncHyp_up_5 comprises the following exons/instrons:
the sequences as shown in SEQ ID NO: 28 to 32, preferably in 5′ to 3′ order: SEQ ID NO: 28, 29, 30, 31, 32; or
the sequences as shown in SEQ ID NO: 33 to 39, preferably in 5′ to 3′ order: SEQ ID NO: 33, 34, 35, 36, 37, 38, 39.

lnc-SERTAD2 comprises the sequence shown in SEQ ID NO: 40.

lnc-GDF7 comprises the sequence shown in SEQ ID NO: 41.

lnc-KPNA2 comprises the sequence shown in SEQ ID NO: 42.

lnc-Meg3 may be selected from a lnRNA comprising any of the sequence shown in SEQ ID NO: 43 to 58.

lnc-RNF151 comprises the sequence shown in SEQ ID NO: 59.

The sequences provided in the sequence listing as part of the description are shown as DNA sequences. However, the person of skill understands that when referring to the actual lncRNA molecule the thymine residues will correspond to uracil at the respective positions in the RNA.

The term “sequence identity” or “percent identical” as applied to nucleic acid molecules is the percentage of nucleic acid residues in a candidate nucleic acid molecule sequence that are identical with a subject nucleic acid molecule sequence, after aligning the sequences to achieve the maximum percent identity, and not considering any nucleic acid residue substitutions as part of the sequence identity. No gaps are introduced into the candidate nucleic acid sequence in order to achieve the best alignment.

When assessing the identity or complementarity of a first and second nucleic acid sequence wherein one sequence is a DNA sequence and the other is an RNA sequence, it must be borne in mind that RNA sequences comprise uracil whereas DNA sequences would comprise thymine as complementary base of adenine. Therefore, whenever in context of the present invention a RNA sequence is referred to any of the sequences in the sequence protocol, these sequences have to be converted into RNA by exchanging thymine with uracil. When assessing sequence identity, an uracil residue is considered to be identical to a thymine residue (because both bind adenine) and when assessing complementarity a uracil residue is considered to be complementary to/capable of forming hydrogen bonds with an adenine residue. In this respect, the present invention when referring to a nucleic acid which is RNA in accordance with the invention contains uracil bases instead of thymine bases, and vice versa. This difference is well recognized in the pertinent art and easily recognized by a person of skill.

Using next generation sequencing, the inventors identified several endothelial long non-coding RNAs (lncRNAs) which regulate endothelial cell function. Atheroprotective laminar flow increased expression of lnc-KPNA2, and lnc-SERTAD2, and down-regulated lnc-GDF7. Inhibition of lnc-KPNA2 and lnc-SERTAD2 reduced angiogenic sprouting suggesting that both lncRNAs exhibit a pro-angiogenic activity. Inhibition of lnc-KPNA2 significantly increased basal and TNFa-induced endothelial adhesion molecule expression whereas inhibition of lnc-SERTAD2 reduced endothelial VCAM1 expression. Lnc-KPNA2 increased expression of key enzymes of the mevalonate pathway indicating a critical role in cholesterol metabolism. In addition the inventors surprisingly show that hypoxia regulates the expression of various lncRNAs and found that the hypoxia-regulated lncRNA Hyp_up_1 is pro-angiogenic, whereas Hyp_down_1 and Meg3 are anti-angiogenic.

The term “endothelial cell dysfunction” refers to a physiological dysfunction of normal biochemical processes carried out by endothelial cells, the cells that line the inner surface of all blood vessels including arteries and veins. For example, endothelial cell dysfunction may result in an inability of blood vessels, such as arteries and arterioles, to dilate normally in response to an appropriate stimulus.

Modulation of LncRNA Function and Expression

The present invention is based on the development of lncRNA as drug targets. Therefore, a central aspect of the invention pertains to the modulation of the expression or function of endothelial lncRNAs as disclosed herein, preferably in the context of a medical treatment. For means or compounds enhancing the expression or function of a lncRNA the present invention will refer to such means or compounds as “agonists” of a respective lncRNA. Alternatively, the expression and/or function of an lncRNA may be reduced or inhibited. In this case the present invention will refer to these mediators of the effect as “antagonists” of the respective lncRNA of the invention. Since lncRNA are RNA-molecules which mediate their biological activity either in the cellular cytoplasm or in the cell nucleus the person of skill can harness all known methods that intervene with the natural RNA metabolism.

In some embodiments an agonist of a lncRNA of the invention is selected from an lncRNA molecule of the invention or a homolog thereof. An lncRNA molecule is an RNA molecule corresponding to a lncRNA sequence as disclosed herein in any of the SEQ ID NO: 1 to 59 as defined herein above (the lncRNA sequences). A homolog in the context of the invention is a nucleic acid, preferably an RNA molecule, which is homologous to any of the lncRNA of the herein described invention, and preferably comprises a sequence of at least 60% sequence identity to any one of the lncRNA sequences shown in SEQ ID NO: 1 to 59 as defined herein above (lncRNA sequences). Further preferred homologs of the invention comprise a nucleic acid sequence that is at least 70%, 80%, 90%, 95%, 96%, 97%, 98%, or most preferably 99% identical to a sequence as shown in any of the SEQ ID NO: 1 to 59 (lncRNA sequences).

Alternatively, the present invention provides expression constructs of lncRNAs as agonists of the invention. An lncRNA expression construct of the invention comprises preferably an expressible sequence of lncRNA of the invention, optionally of homologs thereof, operatively linked to a promoter sequence. Since expression constructs may be used both to express an agonist or an antagonist of an lncRNA of the invention a detailed description of expression constructs is provided herein below.

In order to impair lncRNA expression/function in accordance with the herein described invention the person of skill may choose any suitable methodology for inhibiting RNA expression. In particular preferred are antisense approaches, which apply sequence complementary nucleic acid polymers (antagonists) which mediate the inhibition or destruction of a target RNAs.

According to some embodiments, an lncRNA of the invention may be targeted using an inhibiting agent or therapeutic—an antagonist of the lncRNA—targeting strategy such as antisense RNA, RNA interference (RNAi), siRNA, esiRNA, shRNA, miRNA, decoys, RNA aptamers, small molecule inhibitors, RNA/DNA-binding proteins/peptides, GapmeRs, LNA molecules or other compounds with different formulations to inhibit one or more physiological actions effected by lncRNA. The antisense antagonists of the invention may either be directly administered or used, or alternatively, may be expressed using an expression construct, for example a construct expressing a miRNA having a sequence complementary to an lncRNA of the invention.

For the inhibition of the lnc-RNA of the invention, it is in certain embodiments preferred to use antisense oligonucleotides in order to impair the lnc-RNA expression or function. Anti-sense oligonucleotides (ASOs) are synthetic nucleic acids that bind to a complementary target and suppress function of that target. Typically ASOs are used to reduce or alter expression of RNA targets—lnc-RNA is one preferred example of an RNA that can be targeted by ASOs. As a general principle, ASOs can suppress RNA function via two different mechanisms of action: 1) by steric blocking, wherein the ASO tightly binds the target nucleic acid and inactivates that species, preventing its participation in cellular activities, or 2) by triggering degradation, wherein the ASO binds the target and leads to activation of a cellular nuclease that degrades the targeted nucleic acid species. One class of “target degrading”. ASOs may be composed of several types of nucleic acids, such as RNA, DNA or PNA.

In order to enhance the half-life of an ASO, modifications of the nucleic acids can be introduced. It is in particular useful to protect the ASO from degradation by cellular endonucleases. One modification that gained widespread use comprised DNA modified with phosphorothioate groups (PS). PS modification of internucleotide linkages confers nuclease resistance, making the ASOs more stable both in serum and in cells. As an added benefit, the PS modification also increases binding of the ASO to serum proteins, such as albumin, which decreases the rate of renal excretion following intravenous injection, thereby improving pharmacokinetics and improving functional performance. Therefore, PS modified ASOs are encompassed by the present invention.

Further modifications target the 3′-end of an ASO molecule, for example “Gapmer” compounds having 2′-methoxyethylriboses (MOE's) providing 2′-modified “wings” at the 3′ and 5′ ends flanking a central 2′-deoxy gap region. ASO modifications that improve both binding affinity and nuclease resistance typically are modified nucleosides including locked nucleic acids (LNA), wherein a methyl bridge connects the 2′-oxygen and the 4′-carbon, locking the ribose in an A-form conformation; variations of LNA are also available, such as ethylene-bridged nucleic acids (ENA) that contain an additional methyl group, amino-LNA and thio-LNA. Additionally, other 2′-modifications, such as 2′-0-methoxyethyl (MOE) or 2′-fluoro (2′-F), can also be incorporated into ASOs. Such exemplary modifications are comprised by the present invention.

In the context of the present invention this means that the term “antisense oligonucleotide” pertains to a nucleotide sequence that is complementary to at least a portion of a target lnc-RNA sequence of the invention. The term “oligonucleotide” refers to an oligomer or polymer of nucleotide or nucleoside monomers consisting of naturally occurring bases, sugars, and intersugar (backbone) linkages. The term also includes modified or substituted oligomers comprising non-naturally occurring monomers or portions thereof, which function similarly. Such modified or substituted oligonucleotides may be preferred over naturally occurring forms because of properties such as enhanced cellular uptake, or increased stability in the presence of nucleases as already mentioned above. The term also includes chimeric oligonucleotides which contain two or more chemically distinct regions. For example, chimeric oligonucleotides may contain at least one region of modified nucleotides that confer beneficial properties (e.g. increased nuclease resistance, increased uptake into cells) as well as the anti-sense binding region. In addition, two or more antisense oligonucleotides may be linked to form a chimeric oligonucleotide.

The antisense oligonucleotides of the present invention may be ribonucleic or deoxyribonucleic acids and may contain naturally occurring bases including adenine, guanine, cytosine, thymidine and uracil. The oligonucleotides may also contain modified bases such as xanthine, hypoxanthine, 2-aminoadenine, 6-methyl, 2-propyl and other alkyl adenines, 5-halo uracil, 5-halo cytosine, 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thiolalkyl guanines, 8-hydrodyl guanine and other 8-substituted guanines, other aza and deaza uracils, thymidines, cytosines, adenines, or guanines, 5-tri-fluoromethyl uracil and 5-trifluoro cytosine.

Other antisense oligonucleotides of the invention may contain modified phosphorous, oxygen heteroatoms in the phosphate backbone, short chain alkyl or cycloalkyl intersugar linkages or short chain heteroatomic or heterocyclic intersugar linkages. For example, the antisense oligonucleotides may contain phosphorothioates, phosphotriesters, methyl phosphonates and phosphorodithioates. In addition, the antisense oligonucleotides may contain a combination of linkages, for example, phosphorothioate bonds may link only the four to six 3′-terminal bases, may link all the nucleotides or may link only 1 pair of bases.

The antisense oligonucleotides of the invention may also comprise nucleotide analogs that may be better suited as therapeutic or experimental reagents. An example of an oligonucleotide analogue is a peptide nucleic acid (PNA) in which the deoxribose (or ribose) phosphate backbone in the DNA (or RNA), is replaced with a polymide backbone which is similar to that found in peptides. PNA analogues have been shown to be resistant to degradation by enzymes and to have extended lives in vivo and in vitro. PNAs also form stronger bonds with a complementary DNA sequence due to the lack of charge repulsion between the PNA strand and the DNA strand. Other oligonucleotide analogues may contain nucleotides having polymer backbones, cyclic backbones, or acyclic backbones. For example, the nucleotides may have morpholino backbone structures. Oligonucleotide analogues may also contain groups such as reporter groups, protective groups and groups for improving the pharmacokinetic properties of the oligonucleotide. Antisense oligonucleotides may also incorporate sugar mimetics as will be appreciated by one of skill in the art.

Antisense nucleic acid molecules may be constructed using chemical synthesis and enzymatic ligation reactions using procedures known in the art based on a given lnc-RNA sequence such as those provided herein. The antisense nucleic acid molecules of the invention, or fragments thereof, may be chemically synthesized using naturally occurring nucleotides or variously modified nucleotides designed to increase the biological stability of the molecules or to increase the physical stability of the duplex formed with mRNA or the native gene, e.g. phosphorothioate derivatives and acridine substituted nucleotides. The antisense sequences may also be produced biologically. In this case, an antisense encoding nucleic acid is incorporated within an expression vector that is then introduced into cells in the form of a recombinant plasmid, phagemid or attenuated virus in which antisense sequences are produced under the control of a high efficiency regulatory region, the activity of which may be determined by the cell type into which the vector is introduced.

In another embodiment, siRNA technology may be applied to inhibit expression of a lnc-RNA of the invention. Application of nucleic acid fragments such as siRNA fragments that correspond with regions in lnc-RNA gene and which selectively target a lnc-RNA may be used to block lnc-RNA expression or function.

SiRNA, small interfering RNA molecules, corresponding to a region in the lnc-RNA sequence are made using well-established methods of nucleic acid syntheses as outlined above with respect to antisense oligonucleotides. Since the structure of target lnc-RNAs is known, fragments of RNA/DNA that correspond therewith can readily be made. The effectiveness of selected siRNA to impair lnc-RNA function or expression, for example via targeted degradation, can be confirmed using a lncRNA-expressing cell line. Briefly, selected siRNA may be incubated with a lncRNA-expressing cell line under appropriate growth conditions. Following a sufficient reaction time, i.e. for the siRNA to bind with lnc-RNA to result in decreased levels of the lnc-RNA, the reaction mixture is tested to determine if such a decrease has occurred, for example via quantitative PCR, northern blotting etc.

Antisense oligonucleotides in accordance with the invention may comprise at least one modification that is incorporated at the terminal end of an antisense oligonucleotide, or between two bases of the antisense oligonucleotide, wherein the modification increases binding affinity and nuclease resistance of the antisense oligonucleotide. In one embodiment, the antisense oligonucleotide comprises at least one modification that is located within three bases of a terminal nucleotide. In another embodiment, the antisense oligonucleotide comprises at least one modification that is located between a terminal base and a penultimate base of either the 3′- or the 5′-end of the oligonucleotide. In another embodiment, the antisense oligonucleotide comprises a modification at a terminal end of the oligonucleotide. In a further embodiment, the antisense oligonucleotide comprises a modification at the terminal end or between the terminal base and the penultimate base of both the 3′- and the 5′-ends of the antisense oligonucleotide. In yet a further embodiment, the oligonucleotide contains a non-base modifier at a terminal end or between the terminal base and the penultimate base at the 5′-end and at the 3′-end.

Pathological Angiogenesis

In some embodiments of the present invention it is preferred that the disease associated with endothelial cell dysfunction is pathological angiogenesis. In this embodiment the compound for use in the treatment of pathological angiogenesis is
a. An antagonist of a long non-coding RNA (lncRNA) selected from the group consisting of lnc-KPNA2, lnc-SERTAD2, and lncHyp_up_1, or
b. An agonist of a lncRNA selected from lncHyp_down_1 and lncMeg3.

The term “pathological angiogenesis” refers to the excessive formation and growth of blood vessels during the maintenance and the progression of several disease states. Examples where pathological angiogenesis can occur are blood vessels (atherosclerosis, hemangioma, hemangioendothelioma), bone and joints (rheumatoid arthritis, synovitis, bone and cartilage destruction, osteomyelitis, pannus growth, osteophyte formation, neoplasms and metastasis), skin (warts, pyogenic granulomas, hair growth, Kaposi's sarcoma, scar keloids, allergic oedema, neoplasms), liver, kidney, lung, ear and other epithelia (inflammatory and infectious processes (including hepatitis, glomerulonephritis, pneumonia), asthma, nasal polyps, otitis, transplantation, liver regeneration, neoplasms and metastasis), uterus, ovary and placenta (dysfunctional uterine bleeding (due to intrauterine contraceptive devices), follicular cyst formation, ovarian hyperstimulation syndrome, endometriosis, neoplasms), brain, nerves and eye (retinopathy of prematurity, diabetic retinopathy, choroidal and other intraocular disorders, leukomalacia, neoplasms and metastasis), heart and skeletal muscle due to work overload, adipose tissue (obesity), endocrine organs (thyroiditis, thyroid enlargement, pancreas transplantation), hematopoiesis (AIDS (Kaposi), hematologic malignancies (leukemias, etc.), tumour induced new blood vessels. The pathological angiogenesis may occur in connection with a proliferative disorder, most preferably in connection with a cancer disease. A cancer may be selected from the group consisting of liver cancer, lung cancer, breast cancer, colorectal cancer, stomach cancer, glioblastoma and melanoma, most preferably wherein the cancer is solid cancer, even more preferably a metastatic solid cancer.

Cardiovascular Diseases

In other preferred embodiments of the present invention the compound of the invention is for use in the treatment of a cardiovascular disease. In this embodiment the compound is
a. An agonist of a long non-coding RNA (lncRNA) selected from the group consisting of lnc-KPNA2, lnc-SERTAD2, and lncHyp_up_1, or
b. An antagonist of a lncRNA selected from lncHyp_down_1 and lncMeg3.

A cardiovascular disease in context of the present invention may be a disease associated with a pathological repressed endothelial cell repair, cell growth and/or cell division or is a disease treatable by improving endothelial cell repair, cell growth and/or cell division. Generally, the term “cardiovascular disease,” as used herein, is intended to refer to all pathological states leading to a narrowing and/or occlusion of blood vessels throughout the body. In particular, the term “cardiovascular disease” refers to conditions including atherosclerosis, thrombosis and other related pathological states, especially within arteries of the heart and brain. Accordingly, the term “cardiovascular disease” encompasses, without limitation, various types of heart disease, as well as Alzheimer's disease and vascular dimension. Also comprised are hypertonic disorders of the lungs, such as pulmonary hypertension PAH and similar diseases.

In preferred embodiments of the invention the cardiovascular disease is selected from the group consisting of acute coronary syndrome, acute lung injury (ALI), acute myocardial infarction (AMI), acute respiratory distress syndrome (ARDS), arterial occlusive disease, arteriosclerosis, articular cartilage defect, aseptic systemic inflammation, atherosclerot-ic cardiovascular disease, autoimmune disease, bone fracture, bone fracture, brain edema, brain hypoperfusion, Buerger's disease, burns, cancer, cardiovascular disease, cartilage damage, cerebral infarct, cerebral ischemia, cerebral stroke, cerebrovascular disease, chemotherapy-induced neuropathy, chronic infection, chronic mesenteric is-chemia, claudication, congestive heart failure, connective tissue damage, contusion, coronary artery disease (CAD), critical limb ischemia (CLI), Crohn's disease, deep vein thrombosis, deep wound, delayed ulcer healing, delayed wound-healing, diabetes (type I and type II), diabetic neuropathy, diabetes induced ischemia, disseminated in-travascular coagulation (DIC), embolic brain ischemia, frostbite, graft-versus-host dis-ease, hereditary hemorrhagic telengiectasiaischemic vascular disease, hyperoxic injury, hypoxia, inflammation, inflammatory bowel disease, inflammatory disease, injured tendons, intermittent claudication, intestinal ischemia, ischemia, ischemic brain disease, ischemic heart disease, ischemic peripheral vascular disease, ischemic placenta, ischemic renal disease, ischemic vascular disease, ischemic-reperfusion injury, laceration, left main coronary artery disease, limb ischemia, lower extremity ischemia, myocardial infarction, myocardial ischemia, organ ischemia, osteoarthritis, osteoporosis, osteosar-coma, Parkinson's disease, peripheral arterial disease (PAD), peripheral artery disease, peripheral ischemia, peripheral neuropathy, peripheral vascular disease, pre-cancer, pulmonary edema, pulmonary embolism, remodeling disorder, renal ischemia, retinal ischemia, retinopathy, sepsis, skin ulcers, solid organ transplantation, spinal cord injury, stroke, subchondral-bone cyst, thrombosis, thrombotic brain ischemia, tissue ischemia, transient ischemic attack (TIA), traumatic brain injury, ulcerative colitis, vascular dis-ease of the kidney, vascular inflammatory conditions, von Hippel-Lindau syndrome, or wounds to tissues or organs.

Treatments

The agonists or antagonists as described herein above are useful in the treatment of the various diseases mentioned above. Therefore the present invention provides the use of the compounds of the invention in a curative or prophylactic medical treatment involving the administration of a therapeutically effective amount of the compound to a subject in need of such a treatment.

The agonists or antagonists described may be used alone or in combination with other methods for treating of the various diseases associated with endothelial cell dysfunction. For example if a subject has been diagnosed with ischemia, the one or more agents described above may be combined with administration of a therapeutically effective amount of a compound that is therapeutically active for the treatment of an ischemic event.

The term “effective amount” as used herein refers to an amount of a compound that produces a desired effect. For example, a population of cells may be contacted with an effective amount of a compound to study its effect in vitro (e.g., cell culture) or to produce a desired therapeutic effect ex vivo or in vitro. An effective amount of a compound may be used to produce a therapeutic effect in a subject, such as preventing or treating a target condition, alleviating symptoms associated with the condition, or producing a desired physiological effect. In such a case, the effective amount of a compound is a “therapeutically effective amount,” “therapeutically effective concentration” or “therapeutically effective dose.” The precise effective amount or therapeutically effective amount is an amount of the composition that will yield the most effective results in terms of efficacy of treatment in a given subject or population of cells. This amount will vary depending upon a variety of factors, including but not limited to the characteristics of the compound (including activity, pharmacokinetics, pharmacodynamics, and bioavailability), the physiological condition of the subject (including age, sex, disease type and stage, general physical condition, responsiveness to a given dosage, and type of medication) or cells, the nature of the pharmaceutically acceptable carrier or carriers in the formulation, and the route of administration. Further an effective or therapeutically effective amount may vary depending on whether the compound is administered alone or in combination with another compound, drug, therapy or other therapeutic method or modality. One skilled in the clinical and pharmacological arts will be able to determine an effective amount or therapeutically effective amount through routine experimentation, namely by monitoring a cell's or subject's response to administration of a compound and adjusting the dosage accordingly. For additional guidance, see Remington: The Science and Practice of Pharmacy, 21st Edition, Univ. of Sciences in Philadelphia (USIP), Lippincott Williams & Wilkins, Philadelphia, Pa., 2005, which is hereby incorporated by reference as if fully set forth herein.

The term “in combination” or “in combination with,” as used herein, means in the course of treating the same disease in the same patient using two or more agents, drugs, treatment regimens, treatment modalities or a combination thereof, in any order. This includes simultaneous administration, as well as in a temporally spaced order of up to several days apart. Such combination treatment may also include more than a single administration of any one or more of the agents, drugs, treatment regimens or treatment modalities. Further, the administration of the two or more agents, drugs, treatment regimens, treatment modalities or a combination thereof may be by the same or different routes of administration.

The term “subject” as used herein means a human or other mammal. In some embodiments, the subject may be a patient suffering or in danger of suffering from a disease as disclosed herein.

In another aspect the present invention also provide antagonists of the lncRNA lnc-KNA2 as described herein above for use in the treatment of cancer. The cancer in this case may be independent from a cancer of endothelial cell origin. The inventors surprisingly identified anti-proliferative effects of antagonists of lnc-KPNA2 against cancer cells. Therefore the invention also pertains to antagonists of lnc-KPNA2 (the antagonists as defined above) for use in cancer treatment. The invention also may pertain to methods of treating a cancerous diseases, comprising the administration of an antagonist of lnc-KPNA2 to a patient in need of such a treatment.

The term “cancer” refers in this aspect to a cellular disorder characterized by uncontrolled or disregulated cell proliferation, decreased cellular differentiation, inappropriate ability to invade surrounding tissue, and/or ability to establish new growth at ectopic sites. The term “cancer” includes solid tumors and bloodborne tumors. The term “cancer” encompasses diseases of skin, tissues, organs, bone, cartilage, blood, and vessels. The term “cancer” further encompasses primary and metastatic cancers.

Expression Constructs

Aspects of the present invention relate to various vehicles comprising the nucleic acid molecules, preferably the antisense or lncRNA molecules, of the present invention. By vehicle is understood an agent with which genetic material can be transferred. Herein such vehicles are exemplified as nucleic acid constructs, vectors, and delivery vehicles such as viruses and cells.

By nucleic acid construct or expression construct is understood a genetically engineered nucleic acid. The nucleic acid construct may be a non-replicating and linear nucleic acid, a circular expression vector, an autonomously replicating plasmid or viral expression vector. A nucleic acid construct may comprise several elements such as, but not limited to genes or fragments of same, promoters, enhancers, terminators, poly-A tails, linkers, markers and host homologous sequences for integration. Methods for engineering nucleic acid constructs are well known in the art (see, e.g., Molecular Cloning: A Laboratory Manual, Sambrook et al., eds., Cold Spring Harbor Laboratory, 2nd Edition, Cold Spring Harbor, N.Y., 1989). Furthermore, the present invention provides modified nucleic acids, in particular chemically modified RNA (modRNA) that can be used directly for the delivery of an lnc-RNA sequence of the invention (Zangi L. et al, “Modified mRNA directs the fate of heart progenitor cells and induces vascular regeneration after myocardial infarction” 2013, Nat Biotechnol). Such modified RNA may be produced by use of 3′-O-Me-m7G(5′)ppp(5′)G cap analogs and is described in Warren L, Manos P D, Ahfeldt T, et al. Highly efficient reprogramming to pluripotency and directed differentiation of human cells with synthetic modified mRNA. Cell Stem Cell. 2011; 7:618-630.

Several nucleic acid molecules may be encoded within the same construct and may be linked by an operative linker. By the term operative linker is understood to refer to a sequence of nucleotides that connects two parts of a nucleic acid construct in a manner securing the expression of the encoded nucleic acids via the construct.

The term promoter will be used here to refer to a group of transcriptional control modules that are clustered around the initiation site for RNA polymerase. The best known example of this is the TATA box, but in some promoters lacking a TATA box, such as the promoter for the mammalian terminal deoxynucleotidyl transferase gene and the promoter for the SV 40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription. Any promoter that can direct transcription initiation of the sequences encoded by the nucleic acid construct may be used in the invention.

An aspect of the present invention comprises the nucleic acid construct wherein the sequence of at least one nucleic acid molecule is preceded by a promoter enabling expression of at least one nucleic acid molecule.

It is a further aspect that the promoter is selected from the group of constitutive promoters, inducible promoters, organism specific promoters, tissue specific promoters and cell type specific promoters. Examples of promoters include, but are not limited to: constitutive promoters such as: simian virus 40 (SV40) early promoter, a mouse mammary tumour virus promoter, a human immunodeficiency virus long terminal repeat promoter, a Moloney virus promoter, an avian leukaemia virus promoter, an Epstein-Barr virus immediate early promoter, a Rous sarcoma virus (RSV) promoter, a human actin promoter, a human myosin promoter, a human haemoglobin promoter, cytomegalovirus (CMV) promoter and a human muscle creatine promoter, inducible promoters such as: a metallothionine promoter, a glucocorticoid promoter, a progesterone promoter, and a tetracycline promoter (tet-on or tet-off), tissue specific promoters such as: HER-2 promoter and PSA associated promoter.

Vectors

An aspect of the present invention comprises the nucleic acid construct as described in any of the above, comprised within a delivery vehicle referred to as vector. A delivery vehicle is an entity whereby a nucleotide sequence can be transported from at least one media to another. Delivery vehicles are generally used for expression of the sequences encoded within the nucleic acid construct and/or for the intracellular delivery of the construct. It is within the scope of the present invention that the delivery vehicle is a vehicle selected from the group of: RNA based vehicles, DNA based vehicles/vectors, lipid based vehicles, virally based vehicles and cell based vehicles. Examples of such delivery vehicles include, but are not limited to: biodegradable polymer microspheres, lipid based formulations such as liposome carriers, coating the construct onto colloidal gold particles, lipopolysaccharides, polypeptides, polysaccharides, pegylation of viral vehicles.

A preferred embodiment of the present invention comprises a virus as a delivery vehicle, where the virus is selected from the non-exhaustive group of: adenoviruses, retroviruses, lentiviruses, adeno-associated viruses, herpesviruses, vaccinia viruses, foamy viruses, cytomegaloviruses, Semliki forest virus, poxviruses, RNA virus vector and DNA virus vector. Such viral vectors are well known in the art.

Another aspect of the present invention then pertains to a method for modulating endothelial cell function comprising contacting an endothelial cell with an agonist or antagonist of a lncRNA selected from the group consisting of lnc-KPNA2, lnc-SERTAD2, lncHyp_up_1, lnc-Hyp_down_1 and lnc-Meg3. The endothelial cell function may be selected from endothelial cell repair, endothelial cell growth and/or endothelial cell division.

Another aspect of the invention further pertains to a method for the generation of vascular tissue material, the method comprising the steps of contacting an endothelial cell with an agonist of a lncRNA selected from the group consisting of lnc-KPNA2, lnc-SERTAD2, and lncHyp_up_1, or an antagonist of a lncRNA selected from lncHyp_down_1 and lncMeg3. The vascular tissue material is preferably a blood vessel.

It is preferred that the method of the invention is an ex-vivo or in-vitro method.

In this aspect the lncRNA agonist or antagonist is preferably a compound as described herein above.

Pharmaceutical Compositions

Yet another aspect of the invention relates to a compositions, pharmaceutical compositions and kits comprising an agonist or antagonist of a long non-coding RNA (lncRNA) selected from the group consisting of lnc-KPNA2, lnc-SERTAD2, lncHyp_up_1, lnc-Hyp_down_1, lnc-Meg3, lncRNF151, Hyp_up_3, Hyp_up_5 and lncGDF7, in accordance with the afore described various aspects of the invention. In one embodiment, the composition comprises a compound, combination or composition as described herein, optionally together with a pharmaceutically acceptable carrier.

As used herein the language “pharmaceutically acceptable carrier” is intended to include any and all solvents, solubilizers, fillers, stabilizers, binders, absorbents, bases, buffering agents, lubricants, controlled release vehicles, nanoparticles, liposomes, diluents, emulsifying agents, humectants, lubricants, dispersion media, coatings, antibacterial or antifungal agents, isotonic and absorption delaying agents, and the like, compatible with pharmaceutical administration. The use of such media and agents for pharmaceutically active substances is well-known in the art. Except insofar as any conventional media or agent is incompatible with the active compound, use thereof in the compositions is contemplated. Supplementary agents can also be incorporated into the compositions. In certain embodiments, the pharmaceutically acceptable carrier comprises serum albumin.

The pharmaceutical composition of the invention is formulated to be compatible with its intended route of administration. Examples of routes of administration include parenteral, e.g., intrathecal, intra-arterial, intravenous, intradermal, subcutaneous, oral, transdermal (topical) and transmucosal administration.

Solutions or suspensions used for parenteral, intradermal, or subcutaneous application can include the following components: a sterile diluent such as water for injection, saline solution, fixed oils, polyethylene glycols, glycerine; propylene glycol or other synthetic solvents; anti-bacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phos-phate buffered saline (PBS). In all cases, the injectable composition should be sterile and should be fluid to the extent that easy syringability exists. It must be stable under the condi-tions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the requited particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, and sodium chloride in the composition. Prolonged absorption of the inject-able compositions can be brought about by including in the composition an agent which delays absorption, for example, aluminum monostearate and gelatin.

Sterile injectable solutions can be prepared by incorporating the active compound (e.g., a neuregulin) in the required amount in an appropriate solvent with one or a combination of ingredi-ents enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active in-gredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Stertes; a glidant such as colloidal silicon dioxide; a sweetening agent such as sucrose or saccharin; or a flavoring agent such as peppermint, methyl salicylate, or or-ange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the pharmaceutical compositions are formulated into ointments, salves, gels, or creams as generally known in the art.

In certain embodiments, the pharmaceutical composition is formulated for sustained or controlled release of the active ingredient. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from e.g. Alza Corporation and Nova Pharmaceuticals, Inc. Liposomal suspensions (including liposomes targeted to infected cells with monoclonal antibodies to viral antigens) or nanoparticles, including those prepared with poly(dl-lactide-co-glycolide), can also be used as pharmaceutically acceptable carriers. These can be prepared according to methods known to those skilled in the art.

It is especially advantageous to formulate oral or parenteral compositions in dosage unit form for ease of administration and uniformity of dosage. Dosage unit form as used herein includes physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. The pharmaceutical compositions can be included in a container, pack, or dispenser together with instructions for administration.

Finally provided is the use of an agonist or antagonist of a long non-coding RNA (lncRNA) selected from the group consisting of lnc-KPNA2, lnc-SERTAD2, lncHyp_up_1, lnc-Hyp_down_1, lnc-Meg3, lncRNF151, Hyp_up_3, Hyp_up_5 and lncGDF7, in the modulation of endothelial cell function.

The present invention will now be further described in the following examples with reference to the accompanying figures and sequences, nevertheless, without being limited thereto. For the purposes of the present invention, all references as cited herein are incorporated by reference in their entireties. In the Figures:

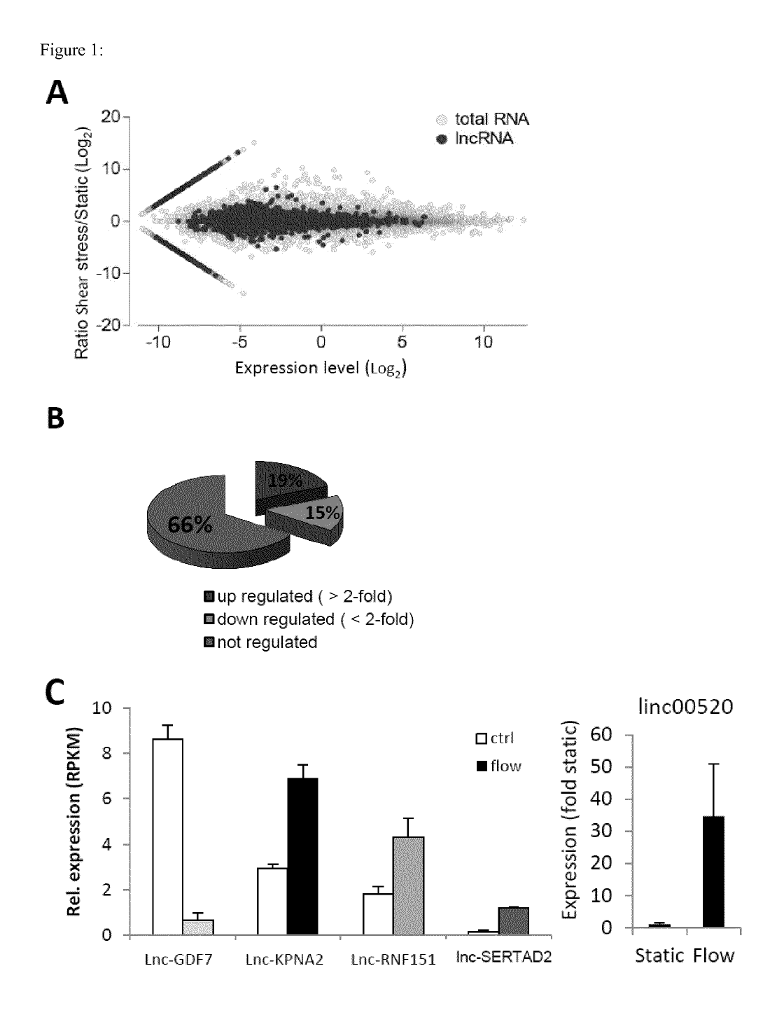

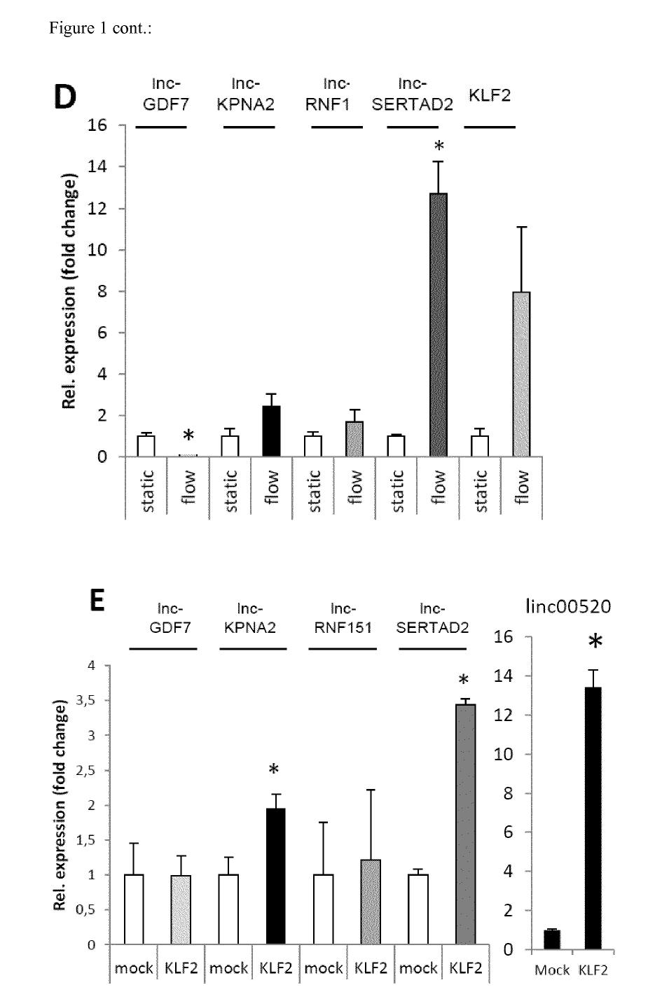

FIG. 1: Regulation of lncRNAs by flow and KLF2. A/B) Expression of coding and non-coding RNAs after exposure of endothelial cells to laminar flow (12 dynes/cm²). C) Relative expression of selected flow-regulated lncRNAs comparing laminar flow and static control conditions. D) qRT-PCR confirming flow-dependent regulation of selected candidates. KLF2 was measured to confirm shear stress stimulation. E) KLF2-dependent regulation of lncRNA candidates. *p<0.05 versus static control.

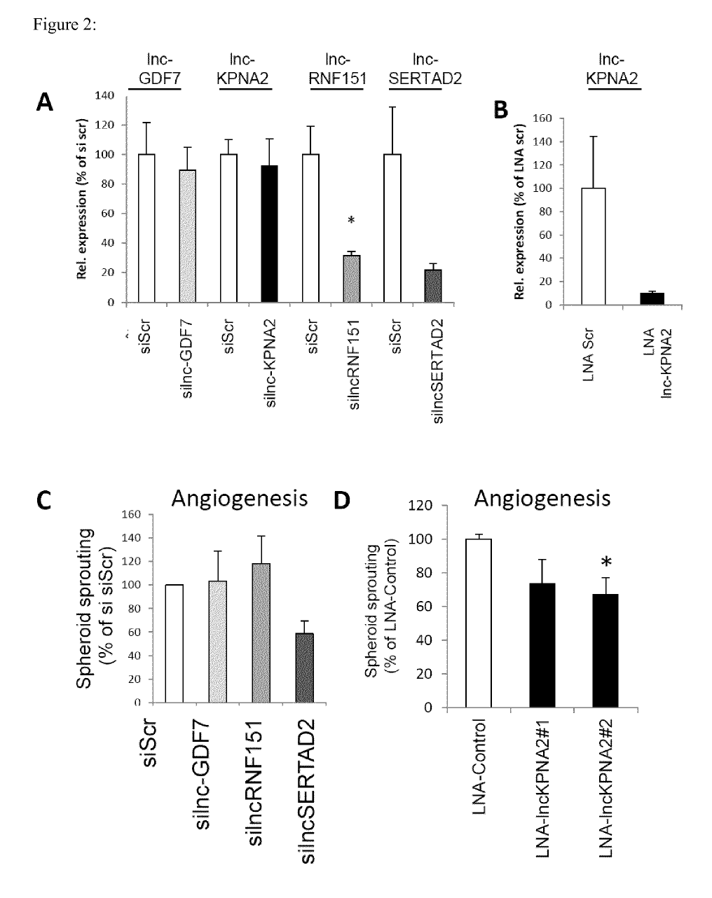

FIG. 2: Angiogenic activity of flow-regulated lncRNAs. lncRNAs were silenced by siRNAs (panel A) or GapmeRs (panel B) and spheroid sprouting was determined (panel C/D).

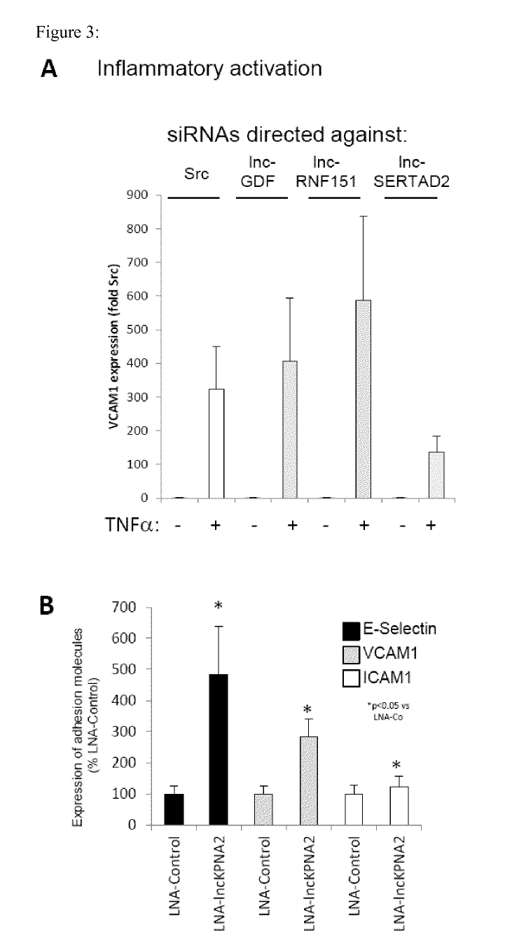

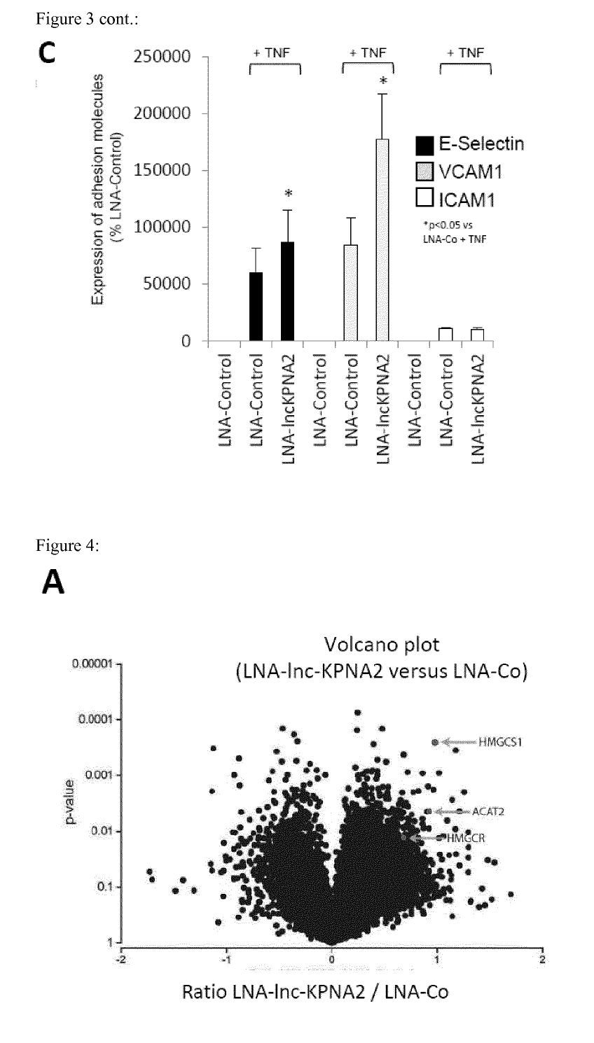

FIG. 3: Regulation of endothelial inflammation by lncRNAs. A) VCAM1 expression after stimulation of siRNA transfected HUVEC with TNFa. B/C) Expression of E-Selectin, VCAM1 and ICAM1 mRNA after silencing of lnc-KPNA2 with GapmeRs. Basal expression is shown in panel B, TNFa-induction is shown in panel C.

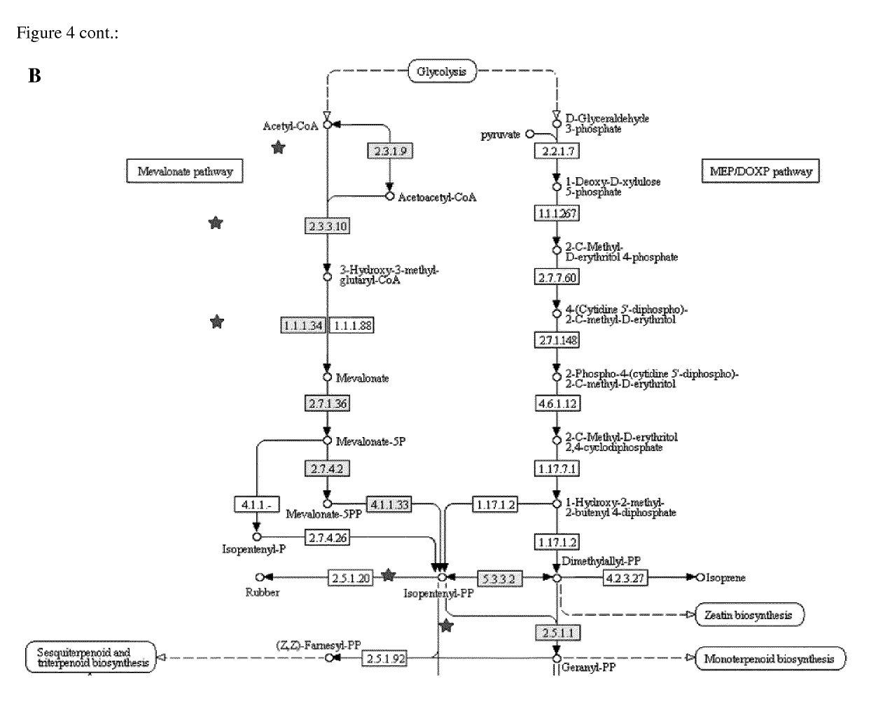

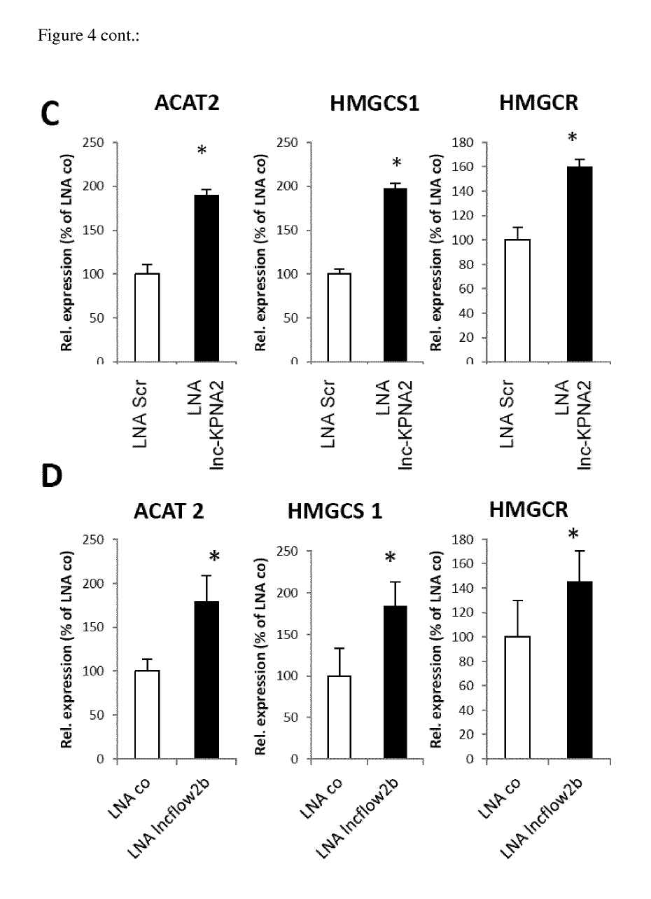

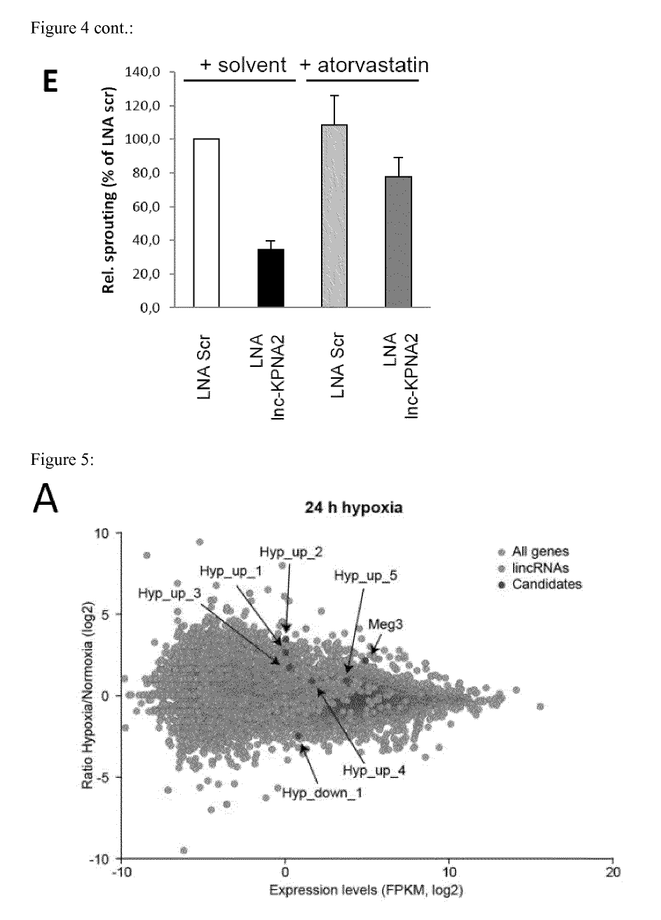

FIG. 4: Gene expression analysis after silencing of lnc-KPNA2. A) Volcano blot showing gene expression changes after silencing of lnc-KPNA2 with GapmeRs. B) Bioinformatic pathway analysis showing regulated genes of the mevalonate pathway after lnc-KPNA2 silencing. C/D) Relative expression analysis of key enzymes of the mevalonate pathway by microarray (C) and validation by qRT-PCR. E) Spheroid sprouting after treatment of GapmeR control or lnc-KPNA2 silenced endothelial cells with or without atorvastatin.

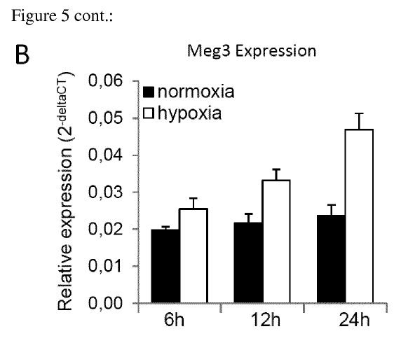

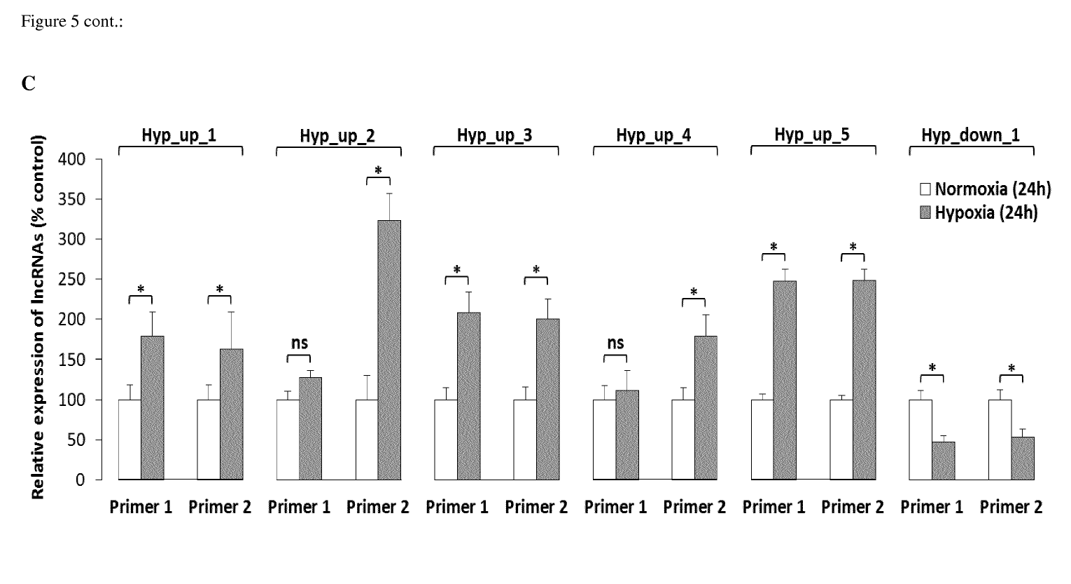

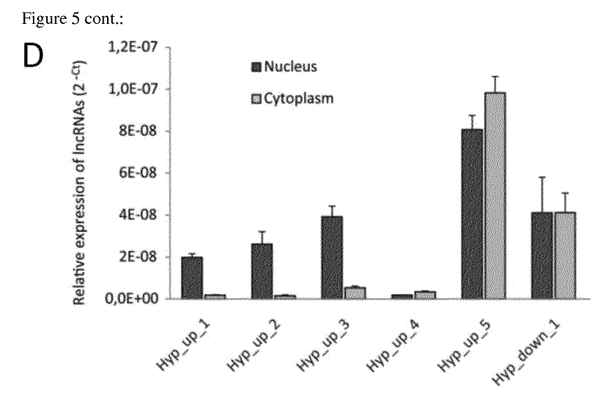

FIG. 5: Regulation, validation and subcellular localization of lncRNAs in HUVEC by hypoxia. A) HUVEC were cultured under hypoxic conditions (0.2% O2) for 24 h or as control under normoxia. RNA was subjected to RNA-sequencing and hypoxia-mediated expression changes are plotted against the relative expression of the transcript. Candidate lncRNAs are highlighted in red. B/C) Validation of candidate lncRNAs by qRT-PCR. B) HUVEC were cultured under normoxic or hypoxic (0.2% O2) conditions for 6 h, 12 h, and 24 h and the relative expression of lnc-Meg3 was analyzed by qRT-PCR. C) HUVEC were cultured under hypoxic conditions (0.2% O2) for 24 h or as control under normoxia. RNA was prepared and analyzed by qRT-PCR using two primer sets for each lncRNA. Data are n=4, *p<0.05, ns=not significant. D) Subcellular distribution of lncRNAs. Nuclear and cytoplasmic fractions were prepared from HUCEC grown under normoxic conditions and corresponding RNA samples were used for qRT-PCR detecting the indicated lncRNAs. Data are n=4.

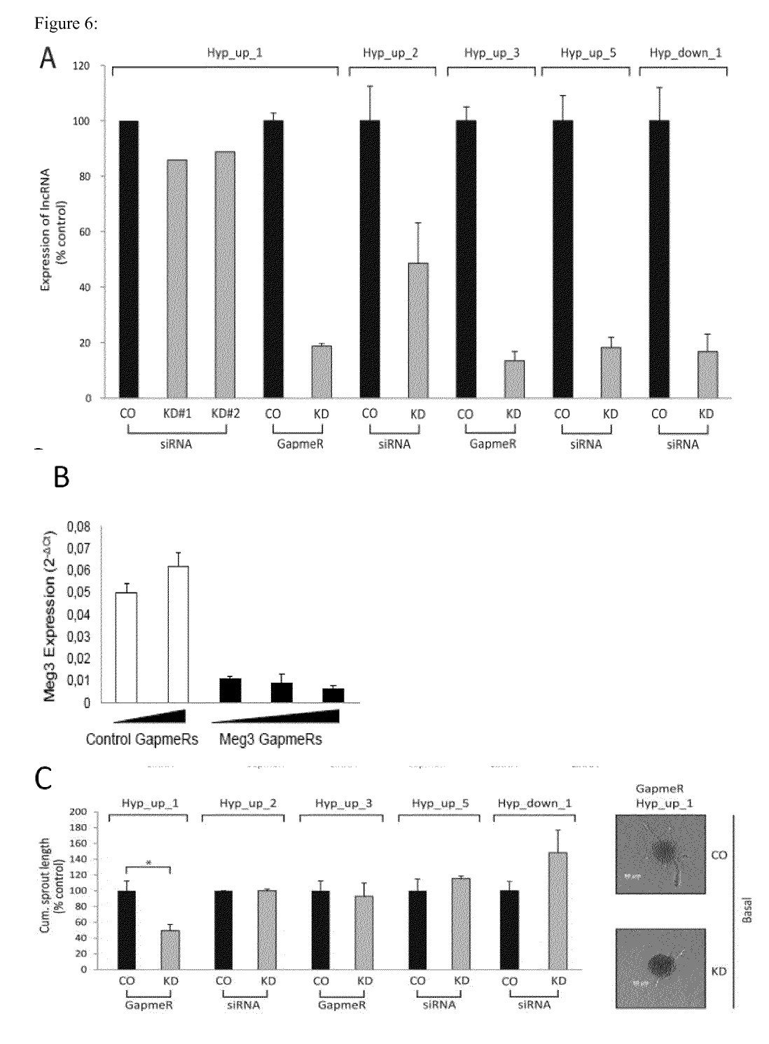

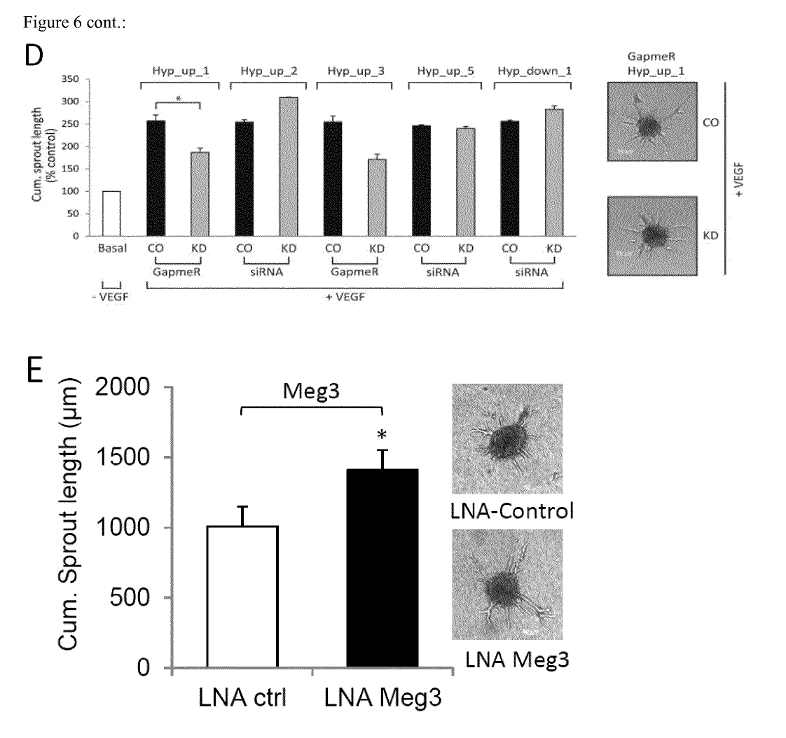

FIG. 6: Effect of hypoxia-regulated lncRNAs on angiogenic sprouting of endothelial cells. A/B) GapmeRs (50 nM) or siRNAs (60 nM) targeting lnc-Hyp_up_1, lnc-Hyp_up_2, lnc-Hyp_up_3, lnc-Hyp_up_5, and lnc-Hyp_down_1 were transfected into HUVEC and lncRNA levels were quantified by qRT-PCR after 48 hours. For lnc-Meg3 silencing, HUVEC were transfected with increasing concentrations (5 to 50 nM) of GapmeRs and lnc-Meg3 expression was quantified by qRT-PCR. C) GapmeRs or siRNAs were transfected 48 hours before spheroids of HUVEC were allowed to sprout under basal conditions in a 3-dimensional matrix for 24 hours. Cumulative sprout length per spheroid was quantified and representative images are shown. D/E) GapmeRs or siRNAs were transfected 48 hours before spheroids of HUVEC were allowed to sprout under VEGF-stimulated conditions (50 ng/ml, 24 h) in a 3-dimensional matrix for 24 hours. Cumulative sprout length per spheroid was quantified and representative images are shown. CO=control siRNAs or GapmeRs; KD=knock-down with siRNAs or GapmeRs as indicated. *p<0.05 versus respective control.

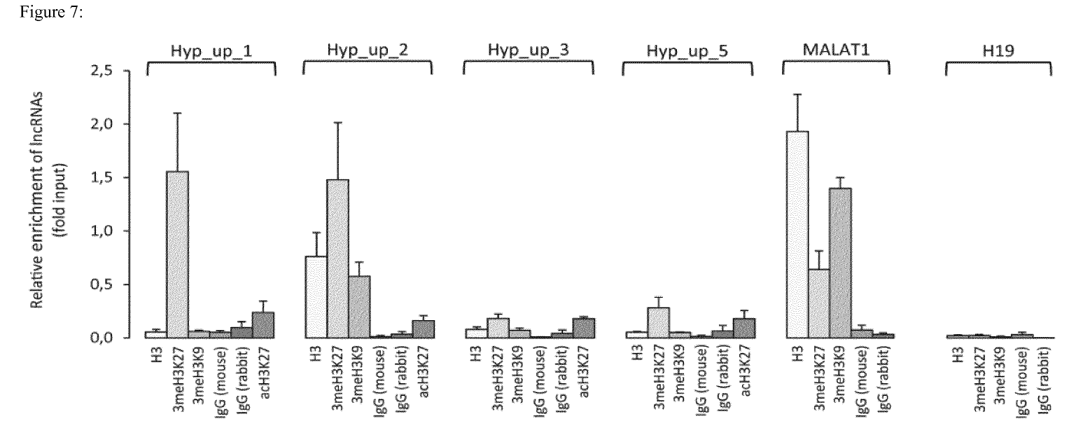

FIG. 7: Histone association of candidate lncRNAs. Cell lysate was prepared from HUCEV cultured under normoxic conditions and used for RNA immunoprecipitations (RIP) using antibodies against histone H3, 3meH3K27, 3meH3K9, and acH3K27. As control, rabbit IgG was used. Co-precipitated lncRNAs (as indicated) were analyzed by qRT-PCR. Data are expressed as fold input. N>3.

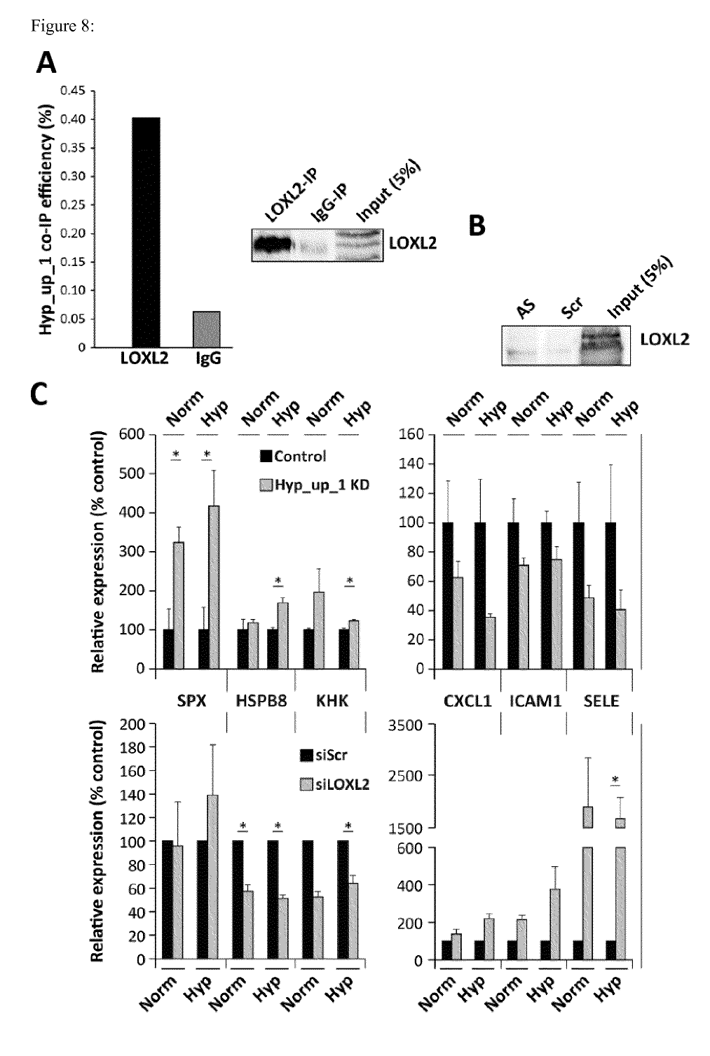

FIG. 8: lncRNA Hyp_up_1 interacts with the lysyl oxidase LOXL2. A) Lox12 was immunoprecipitated from HUCEV cell lysate and co-precipitated RNA was analyzed by qRT-PCR using primer targeting Hyp_up_1. IgG coated beads were used as control. B) Hyp_up_1 was affinity selected by 2′O methyl RNA antisense affinity purification from HUVEC cell lysate and co-purified proteins were subjected to LOXL2 western blotting. Scrambled selection was used as control. C) Regulation of target genes by Hyp_up_1 silencing (upper panel) and LOXL2 silencing (lower panel). HUVEC were silenced for Hyp_up_1 or LOXL2 and grown under normoxic or hypoxic conditions. mRNA expression levels were determined by qRT-PCR using primers targeting Spexin (SPX), Heat shock protein beta-8 (HSPB8), Ketohexokinase (KHK), Growth-regulated alpha protein (CXCL1), Intercellular adhesion molecule 1 (ICAM1), and E-selectin (SELE). *p<0.05 versus respective control.

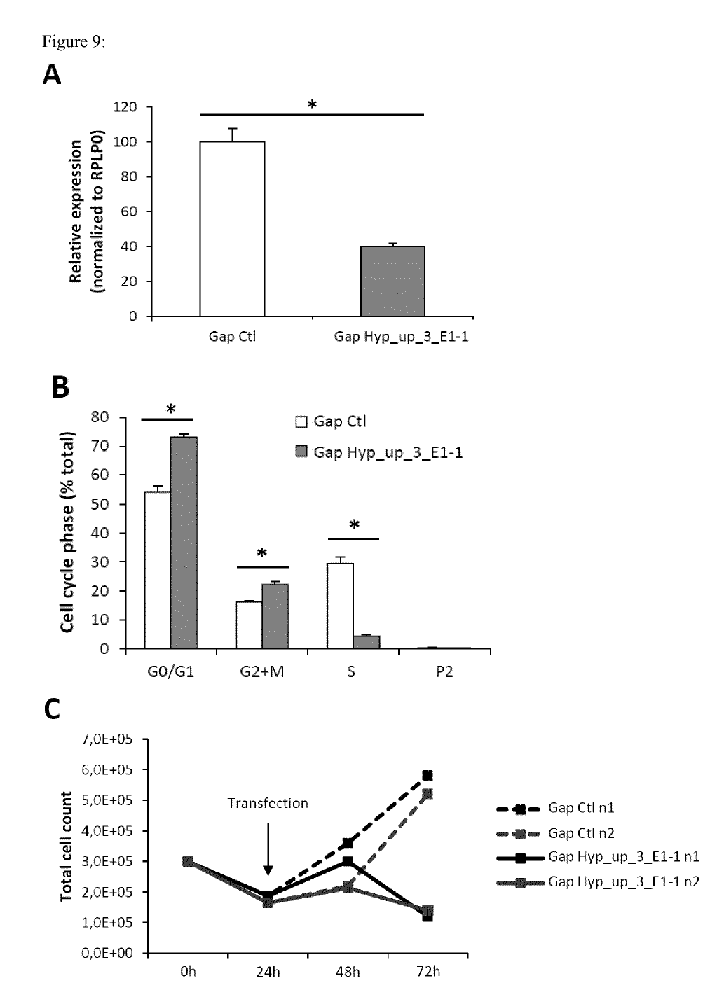

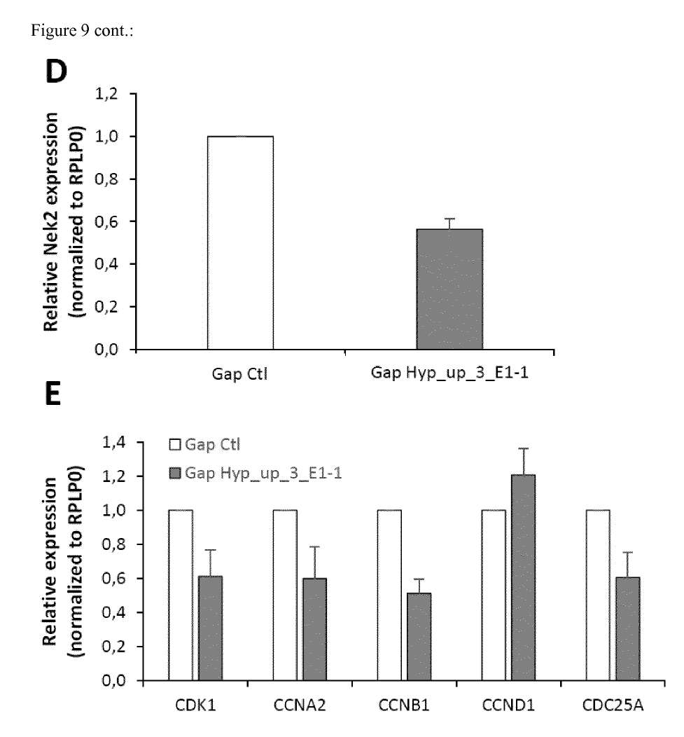

FIG. 9: lncRNA Hyp_up_3 regulates cell cycle progression in HUVEC A) GapmeRs (50 nM) targeting Hyp_up_3 were transfected into HUVEC and expression levels were quantified by qRT-PCR. B) Cell cycle progression was determined by BrdU staining in Hyp_up_3-silenced HEVEC and as control in control transfections. C) 3×105 HUVEC were seeded (0 h) and transfected with GapmeRs targeting Hyp_up_3 (24 h). In the fowling two days, cell numbers were determined (48 h, 72 h). D) Downregulation of the protein kinase Nek2 upon silencing of Hyp_up_3. E) Regulation of the cell cycle genes cyclin-dependant kinase 1 (CDK1), cyclin A2 (CCNA2), cyclin B1 (CCNB1), cyclin D1 (CCND1), and M-phase inducer phosphatase 1 (CDC25A) by silencing of Hyp_up_3. *p<0.05 versus respective control.

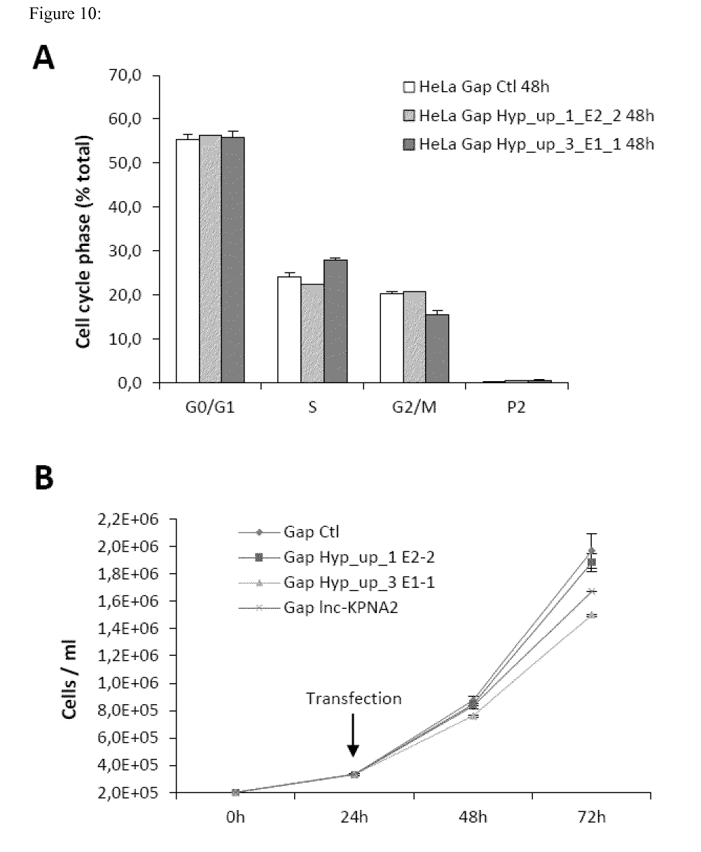

FIG. 10: lncRNA Hyp_up_3 regulates S to G2/M phase transition in HeLa cells A) GapmeRs (50 nM) targeting Hyp_up_1 or Hyp_up_3 were transfected into HeLa cells and cell cycle progression was determined after 48 h of knockdown by BrdU staining. Data is n=2. B) 2×105 HeLa cells were seeded (0 h) and transfected with GapmeRs targeting Hyp_up_1, Hyp_up_3, and lnc-KPNA2 (24 h). In the fowling two days, cell numbers were determined (48 h, 72 h). Data is n=2.

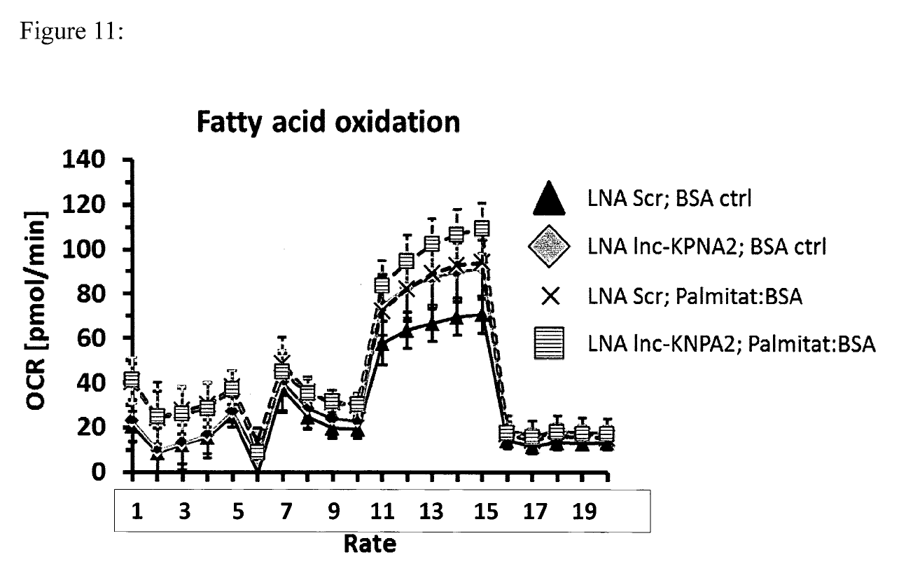

FIG. 11: lnc-KPNA2 controls endothelial cell metabolism. Fatty acid oxidation as measured by Seahorse analysis after silencing of lnc-KPNA2.

SEQ ID NO 1-59 show the sequences of the lncRNA of the invention.

### EXAMPLES

### Example 1

### Laminar Flow Regulated LncRNAs

To identify flow-induced lncRNAs, the inventors compared RNA sequencing data from HUVEC that were cultured under static conditions or were exposed to laminar shear stress for 72 h (20 dynes/cm²). As expected, expression levels of lncRNAs were lower compared to coding RNAs (FIG. 1A). However, the change in gene expression was similar between coding and non-coding transcripts. Flow induced a >2-fold up-regulation of 19% of all lncRNAs analyzed (FIG. 1B), whereas 15% of lncRNAs decreased more than 2-fold. For further analysis, the inventors selected 5 candidates which are highly expressed in HUVECs, and of which 4 were increased by >1.5-fold and one was significantly suppressed by flow (FIG. 1C/D). The flow dependent regulation of the selected lncRNAs was confirmed by qRT-PCR (FIG. 1D). The inventors determined whether the selected lncRNAs are regulated by the flow-induced transcription factor KLF2 and found that lnc-KPNA2, lnc-SERTAD2 and lnc-00520 are induced after KLF2 overexpression in HUVECs.

### Example 2

### LncRNA Regulate Endothelial Cell Function

The function of the selected lncRNAs was next studied by using siRNAs, which reduced expression of the targeted lnc-RNF151 and lnc-SERTAD2 (FIG. 2A). Furthermore, lnc-KPNA2 was targeted by GapmeRs, which are RNase H-inducing antisense oligonucleotides (FIG. 2B). Endothelial cell function was measured by using the spheroid outgrowth assay (FIG. 2C/D). Silencing of lnc-SERTAD2 and lnc-KPNA2 reduced spheroid sprouting, whereas silencing of lnc-RNF151 had no effect. Since flow elicits a profound anti-inflammatory effect in endothelial cells, the inventors additionally determined the effect of lncRNAs on TNFa-induced inflammatory activation of ECs (FIG. 3A). The inventors show that silencing of lnc-SERTAD2 reduces TNFa-induced expression of VCAM1, whereas silencing of lnc-RNF151 did not affect the inflammatory response. Since lnc-KPNA2 was highest expressed at baseline and showed a significant effect on EC functions, the inventors selected this lncRNA for further functional analysis.

### Example 3

### Lnc-KPNA2 Regulates the Mevalonate Pathway

To determine the molecular mechanism by which lnc-KPNA2 controls EC functions, the inventors performed gene expression profiling using microarrays (FIG. 4A). KEGG analysis showed that metabolic pathways are critically regulated by lnc-KPNA2 silencing (FIG. 4B). Of note, the mevalonate pathway, which is targeted by statins and critically controls EC functions, was significantly regulated by silencing of lnc-KPNA2 (FIG. 3B). Particularly, aceto-acetyl-CoA transferase (ACAT2), HMG-CoA-synthase (HMGCS1), and HMG-CoA-reductase (HMGCR) were significantly increased after lnc-KPNA2 silencing (FIG. 4C). These results were confirmed by qRT-PCR (FIG. 4D). Since statins mediate their endothelial protective effect at least in parts by blocking the HMG-CoA-reductase, the inventors investigated whether the pharmacological inhibition of this enzyme may rescue the impaired function of lnc-KPNA2-silenced ECs. Indeed, atorvastatin partially reversed the inhibition of spheroid sprouting after lnc-KPNA2 GapmeR-treatment (FIG. 4E).

In summary laminar flow regulates several lncRNAs, of which lnc-KPNA2 exhibit proangiogenic and anti-inflammatory activities in cultured endothelial cells Inhibition of lnc-KPNA2 reduced spheroid sprouting and increased basal and TNFa-induced expression of adhesion molecules Inhibition of lnc-SERTAD2 reduced spheroid sprouting and TNF-induced expression of VCAM1 suggesting that this lncRNA also promotes angiogenesis but enhances endothelial inflammatory activation.

### Example 4

### Hypoxia-Regulated LncRNAs

To systematically determine the regulation of lncRNAs by hypoxia, the inventors performed deep sequencing of RNA from endothelial cells after exposure to hypoxia (0.2%) for 12 or 24 h. The inventors showed that 71 noncoding RNAs were significantly (p<0.01) up-regulated and 78 lncRNAs were down-regulated after 24 h hypoxia. Based on expression levels (FPKM >1) and regulation by hypoxia, the inventors selected 6 uncharacterized candidates named lnc-Hyp_up_1 to lnc-Hyp_up_5 and lnc-Hyp_down_1 as well as lnc-Meg3 for further analysis (FIG. 5A/B). Of the 6 candidates, 3 are annotated as lincRNAs and 3 are annotated as antisense transcripts. However, the antisense transcripts are not all complementary sequences to the mRNA. For example, lnc-Hyp_up_1 is transcribed from the reverse strand of the GATA6 locus, but it does not overlap with GATA6 mRNA. The inventors confirmed the up-regulation of lnc-Hyp_up 1, lnc-Hyp_up_3 and lnc-Hyp_up_5 and the down-regulation of lnc-Hyp_down_1 by using two sets of primers targeting different regions of the lncRNAs (FIG. 5C). Only one of the two primer sets showed an increased expression of lnc-Hyp_up_2 and lnc-Hyp_up_4 (FIG. 5C), suggesting that different iso forms may exist for these two lncRNAs. For lnc-Meg3 the inventors confirmed up-regulation by using a hypoxia time course experiment, ranging from 6 h to 24 h (FIG. 5B). Depending on their subcellular localization, lncRNAs may comprise different functions. Whereas nuclear lncRNAs can change gene expression levels by altering chromatin states through recruitment chromatin modifying enzymes or modify alternative splicing, cytoplasmic localized lncRNAs may act as sponges for miRNAs or proteins. To gain first insights into the putative mode of action, the inventors determined the nuclear versus cytoplasmic localization of the hypoxia-regulated lncRNAs. The inventors showed that lnc-Hyp_up_1, lnc-Hyp_up_2, and lnc-Hyp_up_3 are predominately localized to the nucleus, whereas the other candidates were detected in the cytoplasm and the nucleus (FIG. 5D).

Hypoxia controls the angiogenic function of endothelial cells. Therefore, it was next investigated whether the candidate lncRNAs affect endothelial cell sprouting as an in vitro measurement for the angiogenic response Inhibition of lnc-Hyp_up_2, lnc-Hyp_up_5 and lnc-Hyp_down_1 was achieved by siRNAs (FIG. 6A, data not shown) and alternatively by LNA™ GapmeRs, which are known to enter the nucleus and induce lncRNA degradation by an RNase H-dependent mechanism. By using GapmeRs the inventors achieved a significant >80% reduction of lnc-Hyp_up_1, lnc-Hyp_up_3, and lnc-Meg3 (FIG. 6A/B). Next, the inventors used the established siRNAs or GapmeRs to determine the biological function of the hypoxia-regulated lncRNAs.

It could be shown that basal sprouting is significantly reduced by silencing of lnc-Hyp_up_1, whereas inhibition of lnc-Hyp_down_1 and lnc-Meg3 increased basal sprouting (FIG. 6C/E). All other tested hypoxia-regulated lncRNAs showed no effect on basal angiogenic sprouting (FIG. 6C). Next it was tested, whether the selected lncRNAs are also involved in VEGF-stimulated angiogenic sprouting of endothelial cells. Endothelial sprouting was significantly decreased after silencing of lnc-Hyp_up_1 and lnc-Hyp_up_3 but was increased by siRNAs directed against lnc-Hyp_down_1 (FIG. 6D).

In order to functionally characterize our selected lncRNAs, their association with histones was determined. In general, acetylation of histones H3 and H4 is associated with increased gene expression, whereas the effect of histone methylation on transcription depends on the lysine acceptor site and the number of methyl groups attached. Methylation can occur at several lysine (K) residues and the residues can be mono, di-, or tri-methylated. Histone H3 modifications at K4 and K36 are associated with activation of gene expression, whereas K27 trimethylation (3meH3K27) and K9 trimethylation (3meH3K9) at promoter sites are repressive. Lnc-Hyp_up_1 and lnc-Hyp_up_2 were enriched in RNA immunoprecipitates of 3meH3K27 but not in precipitates of acetylated H3K27 (acH3K27) or 3meH3K9 (FIG. 7), suggesting that these lncRNAs are predominantly associated with repressive 3meH3K27 chromatin marks and may be involved in epigenetic gene silencing. Controls with other known non-coding RNAs such as lnc-MALAT1, a nuclear enriched lncRNA, which predominantly interacts with H3 and H3K9me3, and H19, which is predominantly localized to the cytoplasm and does not interact with histone complexes, confirm that the association of the newly identified hypoxia-regulated lncRNAs with modified histones appears to be specific (FIG. 7).

In summary it was shown that 1) lnc-Hyp_up_1 is sufficiently expressed (FPKM >1) under baseline conditions, 2) is significantly up-regulated by hypoxia, 3) controls angiogenic sprouting in vivo, 4) is highly enriched in the nuclear fraction, and 5) is strongly associated with silencing 3meH3K27 histone complexes suggesting a putative epigenetic function. In contrast, lnc-Hyp_down_1 and lnc-Meg3 exhibit an anti-angiogenic activity as demonstrated by the increased sprouting observed after silencing of these lncRNAs.

### Example 5

### Hyp_up_1 Interacts and Regulated LoxL2

By pull down assays followed by mass spectrometry, it is shown that Hyp_up_1 interacts with LoxL2 (FIG. 8A/B). LoxL2 is a deaminase, which controls important physiological and pathophysiological processes including the regulation of matrix remodeling (a process e.g. involved in fibrosis) and epithelial-mesenchymal transition (EMT; a process involved in metastasis). Interestingly, it could be shown that Hyp_up_1 inversely regulate LoxL2 targeted genes (FIG. 8C). Since fibrosis and EMT have important functions beyond endothelial cells, these findings indicate that Hyp_up_1 is involved in other pathophysiological processes as well.

### Example 6

### Hyp_up_3 Profoundly Controls Cell Cycle

The function of Hyp_up_3 was further characterized and it is shown that Hyp_up_3 profoundly regulates cell cycle progression and proliferation of endothelial cells (FIG. 9A-C) by interfering with the regulation of the neighboring gene Nek2 (FIG. 9D). Stimulated by these findings, the inventors further explored the role of Hyp_up_3 in tumor cells and, indeed, the inhibition of Hyp_up_3 inhibits tumor cell proliferation (FIG. 10A-B). Thus, Hyp_up_3 is a tumor promoting gene (oncogene).

### Example 7

### lnc-KPNA2 Affects Cellular Metabolism and Influences Tumor Cell Pro-Liferation

Further the mechanism by which lnc-KPNA2 affects endothelial cell functions was investigated. It is shown that inhibition of lnc-KPNA2 profoundly affects endothelial cell metabolism. Further analysis revealed that lnc-KPNA2 particularly regulates fatty acid metabolism (FIG. 11), but had no major effect on glycolysis or mitochondrial oxidation Inhibition of lnc-KPNA2 also slightly reduced tumor cell proliferation (FIG. 10B).

1. A method of treating a disease associated with endothelial cell dysfunction in a subject, comprising the step of administering to the subject a composition that includes a compound that is an agonist or an antagonist of a long non-coding RNA (lncRNA) selected from the group consisting of lnc-KPNA2, lnc-SERTAD2, lnc-Hyp_up_1, lnc-Hyp_down_1, lnc-Meg3, lnc-RNF151, lnc-Hyp_up_3, lnc-Hyp_up_5, and lnc-GDF7.

2. The method of claim 1, wherein the subject administered the composition has a disease associated with endothelial cell dysfunction that is pathological angiogenesis, and wherein the composition is
an antagonist of a long non-coding RNA (lncRNA) selected from the group consisting of lnc-KPNA2, lnc-SERTAD2, and lnc-Hyp_up_1, or an agonist of an lncRNA selected from lnc-Hyp_down_1 and lnc-Meg3.

3. The method of claim 2, wherein the pathologic angiogenesis in the subject administered the composition occurs in connection with a proliferative disorder and wherein the proliferative disorder is a cancer.

4. The method of claim 1, wherein the subject administered the composition has a disease associated with endothelial cell dysfunction that is a cardiovascular disease, and wherein the composition is
an agonist of a long non-coding RNA (lncRNA) selected from the group consisting of lnc-KPNA2, lnc-SERTAD2, and lnc-Hyp_up_1, or an antagonist of an lncRNA selected from lnc-Hyp_down_1 and lnc-Meg3.

5. The method of claim 4, wherein the cardiovascular disease in the subject administered the composition is a disease associated with a pathological repressed endothelial cell repair, cell growth and/or cell division or is a disease treatable by improving endothelial cell repair, cell growth and/or cell division.

6. The method of of claim 1, wherein an lncRNA agonist is selected from the group of an lncRNA molecule, or a homolog thereof or an lncRNA expression construct, or a molecule that when contacted with a cell induces or increases the expression and or function of the lncRNA; and/or wherein an antagonist of a lncRNA is selected from an antagonistic peptide, an antagonistic small molecule, or an inhibitory nucleic acid, an antisense oligonucleotide, a miRNA, siRNA, dsRNA, LNA, GapmeR, or a molecule that represses the expression and/or function of a lncRNA.

7. An in-vitro or ex-vivo method for modulating endothelial cell function, comprising the step of contacting an endothelial cell with an agonist or antagonist of a lncRNA selected from the group consisting of lnc-KPNA2, lnc-SERTAD2, lnc-Hyp_up_1, lnc-Hyp_down_1 and lnc-Meg3.

8. The method according to claim 7, wherein the endothelial cell function is a function selected from endothelial cell repair, endothelial cell growth and/or endothelial cell division.

9. An in-vitro or ex-vivo method for the generation of vascular tissue material, comprising the step of contacting an endothelial cell with an agonist of a lncRNA, wherein the lncRNA is selected from the group consisting of lnc-KPNA2, lnc-SERTAD2, and lnc-Hyp_up_1, or an antagonist of a lncRNA selected from lnc-Hyp_down_1 and lnc-Meg3.

10. The method according to claim 9, wherein the vascular tissue material is a blood vessel.

11. The method according to any of claim 9, wherein an lncRNA agonist is selected from the group of an lnc-RNA molecule, a chemically modified lnc-RNA molecule, or an lncRNA expression construct, or a molecule that when contacted with a cell induces or increases the expression and or function of the lncRNA.

12. The method according to any one of claim 9, wherein an antagonist of a lncRNA is selected from an inhibitory nucleic acid, such as a miRNA, siRNA, dsRNA, LNA, GapmeR, or a molecule that represses the expression and/or function of a lncRNA.

13. A pharmaceutical composition comprising an agonist or an antagonist of a long non-coding RNA (lncRNA) selected from the group consisting of lnc-KPNA2, lnc-SERTAD2, lnc-Hyp_up_1, lnc-Hyp_down_1, lnc-Meg3, lnc-RNF151, lnc-Hyp_up_3, lnc-Hyp_up_5 and lnc-GDF7.

14. A method of treating a disease associated with endothelial cell dysfunction in a subject, comprising the step of administering to the subject a composition that includes a compound that is an agonist or an antagonist of a long non-coding RNA (lncRNA) selected from the group consisting of lnc-KPNA2, lnc-SERTAD2, lnc-Hyp_up_1, lnc-Hyp_down_1, lnc-Meg3, lnc-RNF151, lnc-Hyp_up_3, lnc-Hyp_up_5, and lnc-GDF7.

15. A method of modulating endothelial cell function, comprising the step of contacting an endothelial cell with an agonist or antagonist of a long non-coding RNA (lncRNA) selected from the group consisting of lnc-KPNA2, lnc-SERTAD2, lnc-Hyp_up_1, lnc-Hyp_down_1, lnc-Meg3, lnc-RNF151, lnc-Hyp_up_3, lnc-Hyp_up_5 and lnc-GDF7.

16. The method of claim 3, wherein the cancer is selected from the group consisting of liver cancer, lung cancer, breast cancer, colorectal cancer, stomach cancer and melanoma.

17. The method of claim 16, wherein the cancer is a solid cancer.

18. The method of claim 17, wherein the solid cancer is a metastatic solid cancer.

19. The pharmaceutical composition of claim 13, further including an acceptable carrier and/or excipient.
